(19) United States
(12) Patent Application Publication
(10) Pub. No.: US 2023/0235319 A1
(43) Pub. Date: Jul. 27, 2023

(54) CRISPR-CAS12A DIRECTED RANDOM MUTAGENESIS AGENTS AND METHODS

(71) Applicants: BAYER AKTIENGESELLSCHAFT, Leverkusen (DE); BAYER CROPSCIENCE LP, West Sacramento, CA (US)

(72) Inventors: Philipp Knyphausen, Köln (DE); Brian Vogler, Medford, MA (US); Wayne Coco, Pulheim (DE); Andre Cohnen, Titz-Hasselsweiler (DE); Florian Richter, Köln (DE); Damian Curtis, Davis, CA (US)

(21) Appl. No.: 18/009,900

(22) PCT Filed: Jun. 9, 2021
(86) PCT No.: PCT/EP2021/065391

§ 371 Date: Dec. 12, 2022

Related U.S. Application Data
(60) Provisional application No. 63/038,631, filed on Jun. 12, 2020.

(51) Int. Cl. C12N 15/10

(52) U.S. Cl. CPC C12N 15/1082; C12N 15/102; C12N 15/1055

(57) ABSTRACT

Disclosed are new nucleic acid base-editing systems comprising fusion proteins comprising a) an RNA-programmable nucleic acid recognition module or other suitable nucleic acid recognition module, b) a light inducible reactive oxygen generator. Further disclosed are methods and kits to modify or mutagenize a target DNA region in prokaryotic or eukaryotic cells or organisms.

### REFERENCE TO SEQUENCE LISTING SUBMITTED ELECTRONICALLY

The official copy of the sequence listing is submitted electronially via EFS-Web as and ASCII-formatted sequence listing from a file named “BHC201014_Sequence_Listing_final.txt” created on Jun. 1, 2021, and having a size of 159 kilobytes, and is filed concurrently with the specification. The sequence listing contained in this ASCII-formatted document is part of the specification and is herein incorporated by reference in its entirety.

### FIELD

The present application relates to the random modification of target nucleic acid stretches using novel fusion proteins, comprising particular nucleic acid recognition module (NARM) and particular reactive oxygen species producer (ROSP) as well as methods to obtain such modifications of target nucleic acids using the above proteins.

### BACKGROUND

EMS (Ethylmethane sulfonate) mutagenesis has been a staple method of inducing genetic variation for plant breeding for decades. However, the inability to target chosen regions of a genome for mutagenesis with EMS and other chemical mutagens still requires random mutation rates to be high and screening through large populations to identify valuable mutations within a target gene.

Base Editing using catalytically inactive CRISPR-associated protein such as Cas9, paired with a guide nucleic acid targeting a nucleic acid sequence and a base conversion enzyme such as cytidine deaminase (JP6206893) have been shown to convert specific base pairs in a target DNA. However, such methods are rather limited with respect to the base pair changes obtainable, such cytidine-to-uridine (C-to-T), and are also rather restricted in the size of their editing window.

Existing methods for introducing mutations in nucleic acid molecules have one or more of the following disadvantages:
a) they cannot be directed to a specific region or gene;
b) they exhibit low mutation rates;
c) they are toxic to the host cell;
d) the nature of the mutations is limited, e.g., only from C to T;
e) the window in which mutations are introduced in targeted approaches is too narrow;
f) they are not tunable;
g) they are not programmable;
h) the product of the method might be considered as a genetic modification by regulators.

It is therefore of particular relevance to design a mutagenesis system that combines the selective nature of a CRISPR-Cas guide RNA complex and the ability to randomly mutate a large number of bases in a broader sequence window targeted by the CRISPR-Cas nuclease guide RNA complex.

It is also known in the art that Mini Singlet Oxygen Generator, miniSOG, when fused to a histone can induce mutations in the DNA of C. elegans (Nat Commun 6, 8868 (2015)). There have also been speculations that miniSOG could be fused to a sequence-specific DNA binding protein to specifically mutagenize its unknown targets (Nat Commun 6, 8868 (2015); GENETICS Jan. 1, 2018, vol. 208, no. 1, 1-18). None of these disclosures however provide suitable solutions to the problems addressed in this invention. They are either not programmable to target any given genetic locus or merely speculative without providing any teaching necessary to solve the above technical problems.

Further, but not addressing the problem of mutagenesis, are some fusions of some fluorescent proteins like the enhanced green fluorescent protein with CRISPR-Cas9 to allow for a visualization of transfected RNP complex in addition to utility in flow cytometry applications (https://www.sigmaaldrich.com/catalog/product/sigma/ecas9gfppr?lang=de&region=DE&cm_sp=Insite-_-prodRecColdxviews-_-prodRecCold10-3; as of Feb. 7, 2020). Optogenetic mutagenesis in Caenorhabditis elegans (using miniSOG).

In one aspect these methods might be of particular relevance for generating microorganisms with improved characteristics.

In another aspect these methods might be of particular relevance for generating novel plant traits or plant variants with improved features, such as for instance improved tolerance against biotic or abiotic stress or a tolerance against herbicides, or altered levels of primary or secondary metabolites.

### SUMMARY OF THE INVENTION

This disclosure demonstrates that using a fusion protein comprising
a. a nucleic acid recognition module (NARM), preferably a catalytically inactive guided-nuclease (e.g., without being limiting, a catalytically inactive CRISPR-associated protein such as Cas9 or Cas12a (cpf-1), paired with a guide nucleic acid (guide RNA) targeting a nucleic acid sequence) and
b. a reactive oxygen species producer (ROSP), preferably a light harvesting protein, such as a fluorescent protein (e.g., without being limiting, a fluorescent protein such as mOrange2 or Pp2FbFP_L30M),
   (allows for the introduction of mutations in or in the vicinity of the region targeted by the NARM. Furthermore, the rate of mutations introduced can be adjusted by the level of activation of the ROSP, e.g., by tuning the intensity of a light source when using a light harvesting protein.

One particular aspect of the invention are methods for inducing one or more modifications in a target nucleic acid molecule, comprising the steps:
a) contacting the target nucleic acid molecule with a fusion protein comprising:
   i) a nucleic acid recognition module (NARM); and
   ii) a protein that generates reactive oxygen species (ROSP);
   iii) an optional a linker peptide between the MARM and the ROSP;
b) in the presence of a guide RNA complementary to one strand of the target nucleic acid molecule, and
c) an activation of the ROSP.

The domains of the polypeptides according to the invention may either be connected directly or via a linker peptide. When two or more linker peptides are present in the domain, the linker peptides may be the same or different. Suitable linker peptides include oligopeptide or polypeptide sequences. Linker peptides may be rigid or flexible, and may contain sites designed to be cleaved by protease activity. Such linker peptides may function to increase stability or folding of the domains, increase expression, enable targeting, or improve other biological activity. Various linker peptides are known in the art. See, e.g., Chen, et al., Adv. Drug Deliv. Rev., 2013, 65(10): 1357-1369. In some embodiments, those sequences allow some flexibility between the domains they connect. In some embodiments, the linker peptides comprise one or more of the amino acid sequences listed in paragraph [0025] of WO 2017/070632 A2.

Nucleic Acid Recognition Module (NARM)

A nucleic acid recognition module according to the invention can specifically bind to a target nucleotide sequence in a selected double-stranded DNA. In an embodiment of the invention, the nucleic acid recognition module is from an RNA-programmable CRISPR-associated nuclease (for example, a CRISPR class 2 type II (Cas9) or CRISPR class 2 type V (Cas12a and Cas12b) nuclease) or a variant thereof, and in a complex with a guide RNA (gRNA) is capable of targeting a fusion protein according to the invention to a target nucleotide sequence in a DNA molecule (Stella et al., Nature Structural Biology, 24 (11), pp. 882-892). In some embodiments, the nucleic acid recognition module is from a Cas9 protein or a variant thereof. In some embodiments, the nucleic acid recognition module is from a Cas9 protein or a variant thereof and comprises two domains associated with nuclease activity, most commonly denoted as (i) a RuvC domain and (ii) an HNH domain. In some embodiments, the nuclease activity of the RuvC domain and/or the HNH domain is attenuated (e.g., inactivated), such as by introducing appropriate mutations (Jinek et al., Science, 2012 Aug. 17; 337(6096): 816-821). In some embodiments, the nucleic acid recognition module is a derivative of a Cas9 protein containing an inactivating mutation in only one of the two nuclease domains, resulting in a nickase Cas9 (nCas9), which cleaves only one of the two strands of the target DNA. In some embodiments, the nucleic acid recognition module is a derivative of a Cas9 protein containing inactivating mutations in both of the nuclease domains, resulting in a nuclease-dead Cas9 (dCas9).

Used in the context of an enzymatic activity, “significantly reduced” means that such enzymatic activity is lower than 10% of the activity of the reference protein, for example, lower than 5%, lower than 2%, lower than 1%, or lower than 0.1% of such reference enzymatic activity.

In some embodiments, the nucleic acid recognition module is a zinc finger protein, for example: an engineered or naturally-occurring zinc finger protein with specific binding activity for a target nucleotide sequence of the DNA molecule, as for example described in Choo et al, Nature, 1994 Dec. 15; 372(6507): 642-645; or an engineered zinc finger nickase (ZFNickases), in which one monomer of a zinc finger nuclease dimer comprises a Fok1 cleavage domain that had its nuclease activity inactivated by one or more introduced mutations, as for example described in Kim et al., Genome Res, 2012 July; 22(7): 1327-33. doi: 10.1101/gr.138792.112. Epub 2012 Apr. 20.

In some embodiments, the nucleic acid recognition module is a TALEN protein, for example: an engineered TAL effector protein with specific binding activity for a target nucleotide sequence of the DNA molecule, as for example described in Moscou, M. J., & Bogdanove, A. J., (2009), Science, 326 (5959): 1501; or an engineered TAL effector nickase (TALENickases), in which one monomer of a TALE nuclease dimer comprises a Fok1 cleavage domain that had its nuclease activity inactivated by one or more introduced mutations, as for example described in Biochem Biophys Res Commun. 2014 Mar. 28; 446(1):261-6.

Preferably, the NARM is a catalytically inactivated or partially inactivated (nickase) variant of Cas12a or Cas9. More preferably it is a nuclease deficient variant of Cas12a (Zetsche et al., Cell, 163:759-771 (2015) and Yamano et al., Mol Cell, 67:633-645 (2017), most preferably it is dLbaCpf1 from Lachnospiraceae bacterium as in SEQ ID NO: 2 or SEQ ID NO: 21.

Reactive Oxygen Species Producer (ROSP)

Particularly useful ROSP proteins to carry out the invention include fluorescent proteins and flavin mononucleotide-binding proteins (e.g., Pp2FbFP_L30M (https://www.fpbase.org/protein/WN1JX/) SOPP3 (https://www.fpbase.org/protein/sopp3/), tagRFP (https://www.fpbase.org/protein/tagrfp/), SuperNova (https://www.fpbase.org/protein/supernova-red/), mOrange2(https://www.fpbase.org/protein/morange2/), and other, light-insensitive ROSP proteins, like the engineered peroxidase APEX2 (Myers, S. A. et al., Nat Methods, 15, 437-439 (2018). https://doi.org/10.1038/s41592-018-0007-1). All fpbase entries were accessed on May 29, 2020.

Particularly prefereably, the ROSP is mOrange2 fluorescent protein or Pp2FbFP_L30M. Most preferably it is Pp2FbFP_L30M.

The methods according to the invention use a suitable lightsource that allows an appropriate excitation of the ROSP. Suitable for many applications is for example a fluorescent light source covering wavelengths between 440 nm to 700 nm. A particularly suitable excitation wavelength for Pp2FbFP_L30M is at around 440 nm, and at around 560 nm for mOrange2.

Preferred embodiments of the invention are or utilize fusion proteins in which the NARM is a catalytically inactive form of Cas12a (cpf-1).

More preferred embodiments of the invention are or utilize fusion proteins in which the NARM is a catalytically inactive form of Cas12a (cpf-1) and the ROSP is mOrange2 fluorescent protein or Pp2FbFP_L30M. Most preferably it is Pp2FbFP_L30M.

Specifically preferred embodiments of the invention are or utilize fusion proteins according to SEQ ID NO: 2 or SEQ ID NO: 21.

Most preferred embodiments of the invention are or utilize fusion proteins according SEQ ID NO: 21.

In some embodiments, a fusion protein according to the invention comprises the amino acid sequence of SEQ ID NO: 2, or a variant amino acid sequence thereof having at least 85%, preferably 90%, more preferably 95%, even more preferably 97%, particularly preferably 98%, more particularly preferably 99% sequence identity to SEQ ID NO: 21.

In some embodiments, a fusion protein according to the invention comprises the amino acid sequence of SEQ ID NO: 21, or a variant amino acid sequence thereof having at least 85%, preferably 90%, more preferably 95%, even more preferably 97%, particularly preferably 98%, more particularly preferably 99% sequence identity to SEQ ID NO: 21.

In some embodiments, a nucleic acid encoding a fusion protein according to the invention comprises a nucleic acid sequence encoding the amino acid sequence of SEQ ID NO: 2 or a variant amino acid sequence thereof.

In some embodiments, a nucleic acid encoding a fusion protein according to the invention comprises the nucleic acid sequence of SEQ ID NO: 1 or 3, or a variant nucleic acid sequence thereof having at least 85% (such as at least any of 86%, 87%, 88%, 89%, 90%, 91%, 92%, 93%, 94%, 95%, 96%, 97%, 98%, 99%, or greater) sequence identity to the respective SEQ ID NO.

Guide RNAs/sgRNAs

The systems, compositions, and methods described herein in some embodiments employ a genome-targeting nucleic acid that can direct the activities of an associated polypeptide (e.g., SEQ ID NO: 2 or variant thereof) to a specific target sequence within a target nucleic acid. In some embodiments, the genome-targeting nucleic acid is an RNA. A genome-targeting RNA is referred to as a “guide RNA” or “gRNA” herein. A guide RNA has at least a spacer sequence that hybridizes to a target nucleic acid sequence of interest and a CRISPR repeat sequence (such a CRISPR repeat sequence is also referred to as a “tracr mate sequence”). In Type II systems, the gRNA also has a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-specific polypeptide such that the guide RNA and site-direct polypeptide form a complex. The genome-targeting nucleic acid provides target specificity to the complex by virtue of its association with the site-specific polypeptide. The genome-targeting nucleic acid thus directs the activity of the site-specific polypeptide.

In some embodiments, the genome-targeting nucleic acid is a double-molecule guide RNA. In some embodiments, the genome-targeting nucleic acid is a single-molecule guide RNA or single guide RNA (sgRNA). A double-molecule guide RNA has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence. A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins. A single-molecule guide RNA (sgRNA) in a Type V system has, in the 5′ to 3′ direction, a minimum CRISPR repeat sequence and a spacer sequence.

Exemplary genome-targeting nucleic acids are described, for example, in WO 2018/002719.

In general, a CRISPR repeat sequence includes any sequence that has sufficient complementarity with a tracr sequence to promote one or more of: (1) excision of a DNA targeting segment flanked by CRISPR repeat sequences in a cell containing the corresponding tracr sequence; and (2) formation of a CRISPR complex at a target sequence, wherein the CRISPR complex includes the CRISPR repeat sequence hybridized to the tracr sequence. In general, degree of complementarity is with reference to the optimal alignment of the CRISPR repeat sequence and tracr sequence, along the length of the shorter of the two sequences. Optimal alignment may be determined by any suitable alignment algorithm and may further account for secondary structures, such as self-complementarity within either the tracr sequence or CRISPR repeat sequence. In some embodiments, the degree of complementarity between the tracr sequence and CRISPR repeat sequence along the 30 nucleotides length of the shorter of the two when optimally aligned is about or more than 25%, 30%, 40%, 50%, 60%, 70%, 80%, 90%, 95%, 97.5%, 99%, or higher. In some embodiments, the tracr sequence is about or more than 5, 6, 7, 8, 9, 10, 11, 12, 13, 14, 15, 16, 17, 18, 19, 20, 25, 30, 40, 50, or more nucleotides in length. In some embodiments, the tracr sequence and CRISPR repeat sequence are contained within a single transcript, such that hybridization between the two produces a transcript having a secondary structure, such as a hairpin. In some embodiments, the transcript or transcribed polynucleotide sequence has at least two or more hairpins.

The spacer of a guide RNA includes a nucleotide sequence that is complementary to a sequence in a target DNA. In other words, the spacer of a guide RNA interacts with a target DNA in a sequence-specific manner via hybridization (e.g., base pairing). As such, the nucleotide sequence of the spacer may vary and determines the location within the target DNA that the guide RNA and the target DNA will interact. The DNA-targeting segment of a guide RNA can be modified (e.g., by genetic engineering) to hybridize to any desired sequence within a target DNA.

In some embodiments, the spacer has a length of from 10 nucleotides to 30 nucleotides. In some embodiments, the spacer has a length of from 13 nucleotides to 25 nucleotides. In some embodiments, the spacer has a length of from 15 nucleotides to 23 nucleotides. In some embodiments, the spacer has a length of from 18 nucleotides to 22 nucleotides, e.g., from 20 to 22 nucleotides.

In some embodiments, the percent complementarity between the DNA-targeting sequence of the spacer and the protospacer of the target DNA is at least 60% (e.g., at least 65%, at least 70%, at least 75%, at least 80%, at least 85%, at least 90%, at least 95%, at least 97%, at least 98%, at least 99%, or 100%) over the 20-22 nucleotides.

In some embodiments, the protospacer is directly adjacent to a suitable PAM sequence on its 3′ end or such PAM sequence is part of the DNA targeting sequence in its 3′ portion.

Modifications of guide RNAs can be used to enhance the formation or stability of the CRISPR-Cas genome editing complex comprising guide RNAs and a Cas endonuclease such as SEQ ID NO: 2. Modifications of guide RNAs can also or alternatively be used to enhance the initiation, stability or kinetics of interactions between the genome editing complex with the target sequence in the genome, which can be used for example to enhance on-target activity. Modifications of guide RNAs can also or alternatively be used to enhance specificity, e.g., the relative rates of genome editing at the on-target site as compared to effects at other (off-target) sites.

Nuclear localization signals (NLS) are polypeptide sequences in a protein that enable transport of the protein into the nucleus of eukaryotic cells. When two or more NLS sequences are present in the protein, the NLS sequences may be the same or different. Various NLS sequences are known in the art and would be apparent to the skilled artisan. For example, NLS sequences are described in WO 2001/038547, the contents of which are incorporated herein by reference for their disclosure of exemplary nuclear localization sequences. Such NLSs include, without limitation, the nucleoplasmin bipartite NLS, the c-myc nuclear localization sequence, and the hRNPAI M9 nuclear localization sequence. Exemplary NLSs include those listed in paragraph [00204] of WO 2017/070632 A2.

### Methods of the Disclosure

Methods of Inducing Random Mutations in a Target DNA Locus

Typically, suitable sequence areas in the DNA of a target organism are either selected on target or sequence information or randomly using random sequence information of the target genome. Based on such information single or collections of guide RNA sequences specific for the target areas are selected. Within the target organisms the fusion proteins are expressed or integrated (e.g., by electroporation) in combination with the guide RNA(s) and during a suitable time interval the ROSP in the fusion proteins is activated using an appropriate activation method, for example using a light source that allows a sufficient activation of such ROSP.

The methods are in particular suitable to perform larger targetd random mutagenesis screens in target organisms like for instance microorganisms or plant cells.

Methods of Editing a Genome

In some embodiments, provided herein is a method of targeting, editing, modifying, or manipulating a target DNA at one or more locations in a cell or in vitro environment, comprising introducing into the cell or in vitro environment (a) a fusion protein according to the invention or nucleic acid encoding a fusion protein according to the invention; and (b) a guide RNA (gRNA) or nucleic acid encoding the gRNA, wherein the gRNA is capable of guiding a fusion protein according to the invention to a target nucleic acid sequence in the target DNA. In some embodiments, the method comprises introducing into the cell or in vitro environment a fusion protein according to the invention. In some embodiments, the method comprises introducing into the cell or in vitro environment nucleic acid encoding a fusion protein according to the invention. In some embodiments, the method comprises introducing into the cell or in vitro environment the gRNA. In some embodiments, the method comprises introducing into the cell or in vitro environment nucleic acid encoding the gRNA. In some embodiments, the gRNA is a single guide RNA (sgRNA). In some embodiments, the method comprises introducing into the cell or in vitro environment one or more additional gRNAs or nucleic acid encoding the one or more additional gRNAs targeting the target DNA.

A gRNA or sgRNA and a fusion protein according to the invention may form a ribonucleoprotein (RNP) complex. The guide RNA provides target specificity to the RNP complex by including a nucleotide sequence that is complementary to a sequence of a target DNA. The BEFP of the RNP complex provides the nucleobase-editing activity. In some embodiments, the RNP complex modifies a target DNA, leading to, for example, conversion of a cytidine base within the target DNA to a thymidine. The target DNA may be, for example, naked (e.g., unbound by DNA associated proteins) DNA in vitro, chromosomal DNA in cells in vitro, chromosomal DNA in cells in vivo, etc.

In some embodiments, the methods described herein employ a fusion protein according to the invention further comprising one or more additional heterologous sequences. In some embodiments, a heterologous sequence can provide for subcellular localization of a fusion protein according to the invention (e.g., a nuclear localization signal (NLS) for targeting to the nucleus; a mitochondrial localization signal for targeting to the mitochondria; a chloroplast localization signal for targeting to a chloroplast; an ER retention signal; and the like). In some embodiments, a heterologous sequence can provide a tag for ease of tracking or purification (e.g., a fluorescent protein, e.g., green fluorescent protein (GFP), YFP, RFP, CFP, mCherry, tdTomato, and the like; a histidine tag, e.g., a 6×His tag; a hemagglutinin (HA) tag; a FLAG tag; a Myc tag; and the like). In some embodiments, the heterologous sequence can provide for increased or decreased stability.

In some embodiments, multiple guide RNAs are used to simultaneously modify different locations on the same target DNA or on different target DNAs. In some embodiments, two or more guide RNAs target the same gene or transcript or locus. In some embodiments, two or more guide RNAs target different unrelated loci. In some embodiments, two or more guide RNAs target different, but related loci. In some embodiments, a fusion protein according to the invention is provided directly as a protein. A BEFP can be introduced into a cell (provided to the cell) by any method; such methods are known to those of ordinary skill in the art.

A method for DNA modification or base editing according to the present disclosure finds use in a variety of applications, which are also provided. Applications include research applications; diagnostic applications; industrial applications; and therapeutic applications.

Host Cells

In an aspect, a target nucleic acid is within a cell. In another aspect, a target nucleic acid is within a prokaryotic cell. In an aspect, a prokaryotic cell is a cell from a phylum selected from the group consisting of prokaryotic cell is a cell from a phylum selected from the group consisting of Acidobacteria, Actinobacteria, Aquificae, Armatimonadetes, Bacteroidetes, Caldiserica, Chlamydie, Chlorobi, Chloroflexi, Chrysiogenetes, Coprothermobacterota, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospirae, Planctomycetes, Proteobacteria, Spirochaetes, Synergistetes, Tenericutes, Thermodesulfobacteria, Thermotogae, and Verrucomicrobia. In another aspect, a prokaryotic cell is an Escherichia coli cell. In another aspect, a prokaryotic cell is selected from a genus selected from the group consisting of Escherichia, Agrobacterium, Rhizobium, Sinorhizobium, and Staphylococcus.

Preferably, such prokaryotic cell is of the genus Bacillus.

More preferably, the cell is a strain of Bacillus subtilis.

In another aspect, a target nucleic acid is within a eukaryotic cell. In a further aspect, a eukaryotic cell is an ex vivo cell. In another aspect, a eukaryotic cell is a plant cell. In another aspect, a eukaryotic cell is a plant cell in culture. In another aspect, a eukaryotic cell is an angiosperm plant cell. In another aspect, a eukaryotic cell is a gymnosperm plant cell. In another aspect, a eukaryotic cell is a monocotyledonous plant cell. In another aspect, a eukaryotic cell is a dicotyledonous plant cell. In another aspect, a eukaryotic cell is a corn cell. In another aspect, a eukaryotic cell is a rice cell. In another aspect, a eukaryotic cell is a sorghum cell. In another aspect, a eukaryotic cell is a wheat cell. In another aspect, a eukaryotic cell is a canola cell. In another aspect, a eukaryotic cell is an alfalfa cell. In another aspect, a eukaryotic cell is a soybean cell. In another aspect, a eukaryotic cell is a cotton cell. In another aspect, a eukaryotic cell is a tomato cell. In another aspect, a eukaryotic cell is a potato cell. In a further aspect, a eukaryotic cell is a cucumber cell. In another aspect, a eukaryotic cell is a millet cell. In another aspect, a eukaryotic cell is a barley cell. In another aspect, a eukaryotic cell is a Brassica cell. In another aspect, a eukaryotic cell is a grass cell. In another aspect, a eukaryotic cell is a Setaria cell. In another aspect, a eukaryotic cell is an Arabidopsis cell. In a further aspect, a eukaryotic cell is an algae cell.

In one aspect, a plant cell is an epidermal cell. In another aspect, a plant cell is a stomata cell. In another aspect, a plant cell is a trichome cell. In another aspect, a plant cell is a root cell. In another aspect, a plant cell is a leaf cell. In another aspect, a plant cell is a callus cell. In another aspect, a plant cell is a protoplast cell. In another aspect, a plant cell is a pollen cell. In another aspect, a plant cell is an ovary cell. In another aspect, a plant cell is a floral cell. In another aspect, a plant cell is a meristematic cell. In another aspect, a plant cell is an endosperm cell. In another aspect, a plant cell does not comprise reproductive material and does not mediate the natural reproduction of the plant. In another aspect, a plant cell is a somatic plant cell.

Additional provided plant cells, tissues and organs can be from seed, fruit, leaf, cotyledon, hypocotyl, meristem, embryos, endosperm, root, shoot, stem, pod, flower, inflorescence, stalk, pedicel, style, stigma, receptacle, petal, sepal, pollen, anther, filament, ovary, ovule, pericarp, phloem, and vascular tissue.

In a further aspect, a eukaryotic cell is an animal cell. In another aspect, a eukaryotic cell is an animal cell in culture. In a further aspect, a eukaryotic cell is a human cell. In a further aspect, a eukaryotic cell is a human cell in culture. In a further aspect, a eukaryotic cell is a somatic human cell. In a further aspect, a eukaryotic cell is a cancer cell. In a further aspect, a eukaryotic cell is a mammal cell. In a further aspect, a eukaryotic cell is a mouse cell. In a further aspect, a eukaryotic cell is a pig cell. In a further aspect, a eukaryotic cell is a bovid cell. In a further aspect, a eukaryotic cell is a bird cell. In a further aspect, a eukaryotic cell is a reptile cell. In a further aspect, a eukaryotic cell is an amphibian cell. In a further aspect, a eukaryotic cell is an insect cell. In a further aspect, a eukaryotic cell is an arthropod cell. In a further aspect, a eukaryotic cell is a cephalopod cell. In a further aspect, a eukaryotic cell is an arachnid cell. In a further aspect, a eukaryotic cell is a mollusk cell. In a further aspect, a eukaryotic cell is a nematode cell. In a further aspect, a eukaryotic cell is a fish cell. In another embodiment the method is carried out in a prokaryotic cell.

Kits

In some embodiments, provided herein are kits for carrying out a method described herein. A kit can include one or more of: a fusion protein according to the invention or nucleic acid encoding a fusion protein according to the invention; and a gRNA or nucleic acid encoding the gRNA. A kit may include a complex that includes two or more of: a fusion protein according to the invention; a nucleic acid encoding a fusion protein according to the invention; a guide RNA; a nucleic acid encoding a guide RNA.

In some embodiments, a kit includes: (a) a fusion protein according to the invention or nucleic acid encoding a fusion protein according to the invention; and (b) a gRNA or nucleic acid encoding the gRNA. wherein the gRNA is capable of guiding a fusion protein according to the invention to a target nucleic acid sequence. In some embodiments, the kit comprises a fusion protein according to the invention. In some embodiments, the kit comprises nucleic acid encoding a fusion protein according to the invention. In some embodiments, the kit comprises the gRNA. In some embodiments, the kit comprises nucleic acid encoding the gRNA. In some embodiments, the kit further comprises one or more additional gRNAs or nucleic acid encoding the one or more additional gRNAs. In some embodiments, the kit further comprises one or more additional reagents, where such additional reagents can be selected from: a buffer for introducing a fusion protein according to the invention into a cell; a wash buffer; a control reagent; a control expression vector or polyribonucleotide; a reagent for in vitro production of a fusion protein according to the invention from DNA, and the like.

In some embodiments of any of the kits described herein, a gRNA (including, e.g., two or more guide RNAs) can be provided as an array (e.g., an array of RNA molecules, an array of DNA molecules encoding the guide RNA(s), etc.) Such kits can be useful, for example, for use in any of the methods described herein.

Components of a kit can be in separate containers; or can be combined in a single container.

Any of the kits described herein can further include one or more additional reagents, where such additional reagents can be selected from: a dilution buffer; a reconstitution solution; a wash buffer; a control reagent; a control expression vector or Polyribonucleotide; a reagent for in vitro production of a fusion protein according to the invention from DNA, and the like.

In addition to above-mentioned components, a kit can further include instructions for using the components of the kit to practice the methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (e.g., associated with the packaging or subpackaging) etc. In some embodiments, the instructions are present as an electronic storage data file present on a suitable computer readable storage medium, e.g., CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

This technique may be useful to generate guided mutagenesis to produce engineered, non-GMO products for brewing and also dairy products, in particular Lactobacillus. The above methods are applied will test this method in eukaryotic S. cerevisiae to validate its application outside bacterial hosts.

### Definitions

The terms “polynucleotide,” and “nucleic acid,” used interchangeably herein, refer to a polymeric form of nucleotides of any length, either ribonucleotides or deoxyribonucleotides. Thus, this term includes, but is not limited to, single-, double-, and multi-stranded DNA and RNA, genomic DNA, cDNA, DNA-RNA hybrids/triple helices, and polymers including purine and pyrimidine bases or other natural, chemically or biochemically modified, non-natural, or derivatized nucleotide bases. The terms “polynucleotide” and “nucleic acid” should be understood to include, as applicable to the embodiments being described, single-stranded (such as sense or antisense) and double-stranded nucleic acids.

“Oligonucleotide” generally refers to single- or double-stranded polynucleotides at least about 5 nucleotides in length, unless otherwise indicated. Oligonucleotides are also known as “oligomers” or “oligos” and may be isolated from genes or chemically synthesized by methods known in the art.

“Genomic DNA” refers to the DNA of a genome of an organism including, but not limited to, the DNA of the genome of a bacterium, fungus, archaeon, protist, virus, plant, or animal.

The term “manipulating” DNA encompasses binding, nicking one strand, or cleaving, e.g., cutting both strands of the DNA; or encompasses modifying or editing the DNA or a polypeptide associated with the DNA. Manipulating DNA can silence, activate, or modulate (either increase or decrease) the expression of an RNA or polypeptide encoded by the DNA, or prevent or enhance the binding of a polypeptide to DNA.

By “hybridizable” or “complementary” or “substantially complementary” it is meant that a nucleic acid (e.g., RNA or DNA) includes a sequence of nucleotides that enables it to non-covalently bind, e.g., form Watson-Crick base pairs and/or G/U base pairs, “anneal”, or “hybridize,” to another nucleic acid in a sequence-specific, antiparallel, manner (e.g., a nucleic acid specifically binds to a complementary nucleic acid) under the appropriate in vitro and/or in vivo conditions of temperature and solution ionic strength. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position a protein-binding segment (dsRNA duplex) of a guide RNA molecule, the position is not considered to be non-complementary, but is instead considered to be complementary.

It is understood in the art that the sequence of a nucleic acid need not be 100% complementary to that of a target nucleic acid to be specifically hybridizable. Moreover, a nucleic acid may hybridize over one or more segments such that intervening or adjacent segments are not involved in the hybridization event (e.g., a loop structure or hairpin structure). A nucleic acid can include at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining non complementary nucleotides may be clustered or interspersed with complementary nucleotides and need not be contiguous to each other or to complementary nucleotides. Percent complementarity between particular stretches of nucleic acid sequences within nucleic acids can be determined routinely using methods known in the art, for example, a BLAST program (basic local alignment search tools) and/or PowerBLAST program (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math. 1981(2) 482-489).

The term “peptide” generally refers to a chain of 50 amino acids or fewer. The terms “polypeptide” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g., with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10-6 M, less than 10-7 M, less than 10-8 M, less than 10-9 M, less than 10-10 M, less than 10-11 M, less than 10-12 M, less than 10-13 M, less than 10-14 M, or less than 10-15 M. “Affinity” refers to the strength of binding, increased binding affinity being correlated with a lower Kd.

By “binding domain” it is meant a protein domain that can bind non-covalently to another molecule. A binding domain can bind to, for example, a DNA molecule (and can be termed a “DNA-binding protein”), an RNA molecule (and can be termed an “RNA-binding protein”) and/or a protein molecule (and can be termed a “protein-binding protein”). In the case of a protein domain-binding protein, the binding domain can bind to itself (forming homo-dimers, homo-trimers, etc.) and/or it can bind to one or more molecules of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A nucleic acid or polypeptide has a certain percent “sequence identity” to another nucleic acid or polypeptide, meaning that, when aligned, that percentage of bases or amino acids are the same, and in the same relative position, when comparing the two sequences. Sequence identity can be determined using a number of different methods. To determine sequence identity, sequences can be aligned using various methods and computer programs (e.g., BLAST, T-COFFEE, MUSCLE, MAFFT, etc.), available over the worldwide-web at sites including ncbi.nlm.nili.gov/BLAST, ebi.ac.uk/Tools/msa/tcoffee, ebi.Ac.Uk/Tools/msa/muscle, mafft.cbrc/alignment/software. See, e.g., Altschul et al. (1990), J. Mol. Biol. 215:403-10. In some embodiments of the disclosure, sequence alignments standard in the art are used according to the disclosure to determine amino acid residues in a fusion protein according to the invention domain that “correspond to” amino acid residues in another polypeptide from which a fusion protein according to the invention domain is derived, e.g., a Cas9 endonuclease. The amino acid residues of a fusion protein according to the invention that correspond to amino acid residues of one or more other polypeptides appear at the same position in alignments of the sequences.

A DNA sequence that “encodes” a particular RNA is a DNA nucleic acid sequence that is transcribed into the RNA. A polydeoxyribonucleotide may encode an RNA (mRNA) containing a sequence that is translated into protein, or a polydeoxyribonucleotide may encode an RNA that is not translated into protein (e.g., tRNA, rRNA, siRNA, miRNA, or guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence is generally located at 3′ of the coding sequence.

As used herein, a “promoter sequence” or “promoter” is a DNA regulatory region capable of binding RNA polymerase and initiating transcription of a downstream (3′ direction) coding or non-coding sequence. As used herein, the promoter sequence is bounded at its 3′ terminus by the transcription initiation site and extends upstream (5 direction) to include the minimum number of bases or elements necessary to initiate transcription at levels detectable above background. Within the promoter sequence is a transcription initiation site, as well as protein binding domains responsible for the binding of RNA polymerase. Eukaryotic promoters often, but not always, contain “TATA” boxes and “CAAT” boxes. Various promoters, including inducible promoters, may be used to drive the various vectors of the present disclosure. A promoter can be a constitutively active promoter (e.g., a promoter that is constitutively in an active “ON” state), it may be an inducible promoter (e.g., a promoter whose state, active/“ON” or inactive/“OFF”, is controlled by an external stimulus, e.g., the presence of a particular temperature, compound, or protein.), it may be a spatially restricted promoter (e.g., transcriptional control element, enhancer, etc. (e.g., tissue specific promoter, cell type specific promoter, etc.), and it may be a temporally restricted promoter (e.g., the promoter is in the “ON” state or “OFF” state during specific stages of embryonic development or during specific stages of a biological process, e.g., hair follicle cycle in mice). Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III, pol IV, and pol V). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like. Examples of inducible promoters include, but are not limited to T7 RNA polymerase promoter, T3 RNA polymerase promoter, isopropyl-beta-D-thiogalactopyranoside (IPTG)-regulated promoter, lactose induced promoter, heat shock promoter, Tetracycline-regulated promoter, steroid-regulated promoter, metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

In some embodiments, the promoter is a spatially restricted promoter (e.g., cell type specific promoter, tissue specific promoter, etc.) such that in a multi-cellular organism, the promoter is active (e.g., “ON”) in a subset of specific cells. Spatially restricted promoters may also be referred to as enhancers, transcriptional control elements, control sequences, etc. Any suitable spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding a site-specific modifying enzyme in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle in mice). For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSEN02, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TH) (see, e.g., Oh et al. (2009) Gene Ther. 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca2+-calmodulin-dependent protein kinase 11-alpha (CamKIM) promoter (see, e.g., Mayford et al. (1996) Proc. Natl. Acad. Sci. USA 93:13250; and Casanova et al. (2001) Genesis 31:37); a CMV enhancer/platelet-derived growth factor-p promoter (see, e.g., Liu et al. (2004) Gene Therapy 11:52-60); and the like.

The terms “DNA regulatory sequences,” “control elements,” and “regulatory elements,” used interchangeably herein, refer to transcriptional and translational control sequences, such as promoters, enhancers, polyadenylation signals, terminators, and the like, that provide for and/or regulate transcription of a nucleic acid sequence (e.g., a sequence encoding a guide RNA or a sequence encoding a fusion protein according to the invention) and/or regulate translation of an encoded polypeptide.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or nucleic acid sequence that is present in an organism (including in a virus) that can be isolated from a source in nature and that has not been intentionally modified by a human in the laboratory is naturally occurring.

“Heterologous,” as used herein, means a nucleotide or peptide that is not found in the native nucleic acid or protein, respectively. A BEFP described herein may comprise the RNA-binding domain of a fusion protein according to the invention (or a variant thereof) fused to a heterologous polypeptide sequence (e.g., a polypeptide sequence from a protein other than BEFP). The heterologous polypeptide may exhibit an activity (e.g., enzymatic activity) that will also be exhibited by a fusion protein according to the invention (e.g., methyltransferase activity, acetyltransferase activity, kinase activity, ubiquitinating activity, etc.) A heterologous nucleic acid may be linked to a naturally-occurring nucleic acid (or a variant thereof) (e.g., by genetic engineering) to generate a fusion nucleic acid encoding a fusion polypeptide. As another example, in a fusion variant BEFP, a variant BEFP may be fused to a heterologous polypeptide (e.g., a polypeptide other than BEFP), which exhibits an activity that will also be exhibited by the fusion variant BEFP. A heterologous nucleic acid may be linked to a variant BEFP (e.g., by genetic engineering) to generate a nucleic acid encoding a fusion variant BEFP. “Heterologous,” as used herein, additionally means a nucleotide or polypeptide in a cell that is not its native cell.

The term “cognate” refers to two biomolecules that normally interact or co-exist in nature.

“Recombinant,” as used herein, means that a particular nucleic acid (DNA or RNA) or vector is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid that is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). In addition or alternatively, a DNA sequence encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination can be accomplished by chemical synthesis means, or by the artificial manipulation of isolated segments of nucleic acids, e.g., by genetic engineering techniques. Such is generally done to replace a codon with a codon encoding the same amino acid, a conservative amino acid, or a non-conservative amino acid. In addition or alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant nucleic acid encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.) Thus, a “recombinant” polypeptide is the result of human intervention, but may be a naturally occurring amino acid sequence. The term “non-naturally occurring” includes molecules that are markedly different from their naturally occurring counterparts, including chemically modified or mutated molecules.

A “vector” or “expression vector” is a replicon, such as plasmid, phage, virus, or cosmid, to which another DNA segment, e.g., an “insert”, may be attached so as to bring about the replication of the attached segment in a cell.

An “expression cassette” includes a DNA coding sequence operably linked to a promoter. “Operably linked” refers to a juxtaposition wherein the components so described are in a relationship permitting them to function in their intended manner. For example, a promoter is operably linked to a coding sequence if the promoter affects its transcription or expression. The terms “recombinant expression vector,” or “DNA construct” are used interchangeably herein to refer to a DNA molecule comprising a vector and at least one insert. Recombinant expression vectors are generally generated for the purpose of expressing and/or propagating the insert(s), or for the construction of other recombinant nucleotide sequences. The nucleic acid(s) may or may not be operably linked to a promoter sequence and may or may not be operably linked to DNA regulatory sequences.

The term “operably linked”, as used herein, denotes a physical or functional linkage between two or more elements, e.g., polypeptide sequences or nucleic acid sequences, which permits them to operate in their intended fashion. For example, an operably linkage between a nucleic acid of interest and a regulatory sequence (for example, a promoter) is functional link that allows for expression of the nucleic acid of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous.

A cell has been “genetically modified” or “transformed” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell.

In prokaryotes, yeast, and mammalian cells, for example, a transforming DNA can be maintained on an episomal element such as a plasmid. With respect to eukaryotic cells, a stably transformed cell is one in which the transforming DNA is integrated into a chromosome so that it is inherited by daughter cells through chromosome replication. This stability is demonstrated by the ability of the eukaryotic cell to establish cell lines or clones that include a population of daughter cells containing the transforming DNA integrated into chromosomal DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include, but are not limited to, e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep. 13. pp: S0169-409X(12)00283-9. doi:10.1016/j.addr.2012.09.023), and the like.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a bacterial host cell is a genetically modified bacterial host cell by virtue of introduction into a suitable bacterial host cell of an exogenous nucleic acid (e.g., a plasmid or recombinant expression vector) and a eukaryotic host cell is a genetically modified eukaryotic host cell (e.g., a mammalian germ cell), by virtue of introduction into a suitable eukaryotic host cell of an exogenous nucleic acid.

A “target DNA” as used herein is a polydeoxyribonucleotide that includes a “target site” or “target sequence.” The terms “target site,” “target sequence,” “target protospacer DNA,” or “protospacer-like sequence” are used interchangeably herein to refer to a nucleic acid sequence present in a target DNA to which a DNA-targeting segment (also referred to as a “spacer”) of a guide RNA can bind, provided permissive conditions for binding exist. For example, the target site (or target sequence) 5-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see, e.g., Sambrook, supra. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “non-complementary strand” or “non-complementary strand.”

By “site-specific modifying enzyme” or “RNA-binding site-specific modifying enzyme” is meant a polypeptide that binds RNA and is targeted to a specific DNA sequence, such as a fusion protein according to the invention. A site-specific modifying enzyme as described herein is targeted to a specific DNA sequence by the RNA molecule to which it is bound. The RNA molecule includes a sequence that binds, hybridizes to, or is complementary to a target sequence within the target DNA, thus targeting the bound polypeptide to a specific location within the target DNA (the target sequence). By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a guide RNA and a site-specific modifying enzyme is used for targeted double-stranded DNA cleavage.

“Nuclease” and “endonuclease” are used interchangeably herein to mean an enzyme that possesses endonucleolytic catalytic activity for nucleic acid cleavage.

By “cleavage domain” or “active domain” or “nuclease domain” of a nuclease it is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

The “guide sequence” or “DNA-targeting segment” or “DNA-targeting sequence” or “spacer” includes a nucleotide sequence that is complementary to a specific sequence within a target DNA (the complementary strand of the target DNA) designated the “protospacer-like” sequence herein. The protein-binding segment (or “protein-binding sequence”) interacts with a site-specific modifying enzyme. When the site-specific modifying enzyme is a fusion protein according to the invention or BEFP-related polypeptide (described in more detail below), site-specific cleavage of the target DNA occurs at locations determined by both (i) base-pairing complementarity between the guide RNA and the target DNA; and (ii) a short motif (referred to as the protospacer adjacent motif (PAM)) in the target DNA. The protein-binding segment of a guide RNA includes, in part, two complementary stretches of nucleotides that hybridize to one another to form a double-stranded RNA duplex (dsRNA duplex). In some embodiments, a nucleic acid (e.g., a guide RNA, a nucleic acid encoding a guide RNA; a nucleic acid encoding a site-specific modifying enzyme; etc.) includes a modification or sequence that provides for an additional desirable feature (e.g., modified or regulated stability; subcellular targeting; tracking, e.g., a fluorescent label; a binding site for a protein or protein complex; etc.) Non-limiting examples include: a 5′ cap (e.g., a 7-methylguanylate cap (m7G)); a 3′ polyadenylated tail (e.g., a 3′ poly(A) tail); a riboswitch sequence (e.g., to allow for regulated stability and/or regulated accessibility by proteins and/or protein complexes); a stability control sequence; a sequence that forms a dsRNA duplex (e.g., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

In some embodiments, a guide RNA includes an additional segment at either the 5′ or 3′ end that provides for any of the features described above. For example, a suitable third segment can include a 5′ cap (e.g., a 7-methylguanylate cap (m7G)); a 3′ polyadenylated tail (e.g., a 3′ poly(A) tail); a riboswitch sequence (e.g., to allow for regulated stability and/or regulated accessibility by proteins and protein complexes); a stability control sequence; a sequence that forms a dsRNA duplex (e.g., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

A guide RNA and a site-specific modifying enzyme such as a fusion protein according to the invention may form a ribonucleoprotein complex (e.g., bind via non-covalent interactions). The guide RNA provides target specificity to the complex by comprising a nucleotide sequence that is complementary to a sequence of a target DNA. The site-specific modifying enzyme of the complex provides the modifying activity. In other words, the site-specific modifying enzyme is guided to a target DNA sequence (e.g., a target sequence in a chromosomal nucleic acid; a target sequence in an extrachromosomal nucleic acid, e.g., an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA. RNA aptamers are known in the art and are generally a synthetic version of a riboswitch. The terms “RNA aptamer” and “riboswitch” are used interchangeably herein to encompass both synthetic and natural nucleic acid sequences that provide for inducible regulation of the structure (and therefore the availability of specific sequences) of the RNA molecule of which they are part. RNA aptamers generally include a sequence that folds into a particular structure (e.g., a hairpin), which specifically binds a particular drug (e.g., a small molecule). Binding of the drug causes a structural change in the folding of the RNA, which changes a feature of the nucleic acid of which the aptamer is a part. As non-limiting examples: (i) an activator-RNA with an aptamer may not be able to bind to the cognate targeter RNA unless the aptamer is bound by the appropriate drug; (ii) a targeter-RNA with an aptamer may not be able to bind to the cognate activator-RNA unless the aptamer is bound by the appropriate drug; and (iii) a targeter-RNA and an activator-RNA, each comprising a different aptamer that binds a different drug, may not be able to bind to each other unless both drugs are present. As illustrated by these examples, a two-molecule guide RNA can be designed to be inducible.

Examples of aptamers and riboswitches can be found, for example, in: Nakamura et al., Genes Cells. 2012 May; 17(5):344-64; Vavalle et al., Future Cardiol. 2012 May; 8(3):371-82; Citartan et al., Biosens Bioelectron. 2012 Apr. 15; 34(1):1-11; and Liberman et al., Wiley lnterdiscip Rev RNA. 2012 May-June; 3(3):369-84; all of which are herein incorporated by reference in their entireties.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

Examples of aptamers and riboswitches can be found, for example, in: Nakamura et al., Genes Cells. 2012 May; 17(5):344-64; Vavalle et al., Future Cardiol. 2012 May; 8(3):371-82; Citartan et al., Biosens Bioelectron. 2012 Apr. 15; 34(1):1-11; and Liberman et al., Wiley lnterdiscip Rev RNA. 2012 May-June; 3(3):369-84; all of which are herein incorporated by reference in their entirety.

The term “stem cell” is used herein to refer to a cell (e.g., plant stem cell, vertebrate stem cell) that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into lineage-restricted progenitor cells (e.g., mesodermal stem cells), which in turn can differentiate into cells that are further restricted (e.g., neuron progenitors), which can differentiate into end-stage cells (e.g., terminally differentiated cells, e.g., neurons. cardiomyocytes, etc.), which play a characteristic role in a certain tissue type, and may or may not retain the capacity to proliferate further. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et al., Science. 1998 Nov. 6; 282(5391):1145-7) whereas induced pluripotent stem cells (iPSCs) are derived from somatic cells (Takahashi et al., Cell. 2007 Nov. 30; 131(5):861-72; Takahashi et al., Nat Protoc. 2007; 2(12):3081-9; Yu et al., Science. 2007 Dec. 21; 318(5858):1917-20. Epub 2007 Nov. 20).

Because the term PSC refers to pluripotent stem cells regardless of their derivation, the term PSC encompasses the terms ESC and iPSC, as well as the term embryonic germ stem cells (EGSC), which are another example of a PSC. PSCs may be in the form of an established cell line, they may be obtained directly from primary embryonic tissue, or they may be derived from a somatic cell. PSCs can be target cells of the methods described herein.

By “embryonic stem cell” (ESC) is meant a PSC that was isolated from an embryo, such as from the inner cell mass of the blastocyst. ESC lines are listed in the NIH Human Embryonic Stem Cell Registry, e.g., hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute). Stem cells of interest also include embryonic stem cells from other primates, such as Rhesus stem cells and marmoset stem cells. The stem cells may be obtained from any mammalian species, e.g., human, equine, bovine, porcine, canine, feline, rodent, e.g., mice, rats. hamster, primate, etc. (Thomson et al., (1998) Science 282:1145; Thomson et al., (1995) Proc. Natl. Acad. Sci. USA 92:7844; Thomson et al., (1996) Biol. Reprod. 55:254; Shamblott et al., Proc. Natl. Acad. Sci. USA 95:13726, 1998). In culture, ESCs generally grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. Nos. 7,029,913, 5,843,780, and 6,200,806, the disclosures of which are incorporated herein by reference. Methods for proliferating hESCs in the undifferentiated form are described in WO 99/20741, WO 01/51616, and WO 03/020920. By “embryonic germ stem cell” (EGSC) or “embryonic germ cell” or “EG cell” is meant a PSC that is derived from germ cells and/or germ cell progenitors, e.g., primordial germ cells, e.g., those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al., (2001) Proc. Natl. Acad. Sci. USA 98: 113; Shamblott, M., et al., (1998) Proc. Natl. Acad. Sci. USA, 95:13726; and Koshimizu, U., et al. (1996) Development, 122:1235, the disclosures of which are incorporated herein by reference.

By “induced pluripotent stem cell” or “iPSC” it is meant a PSC that is derived from a cell that is not a PSC (e.g., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, Fox03, GDF3, Cyp26al, TERT, and zfp42.

Examples of methods of generating and characterizing iPSCs may be found in, for example, U.S. Patent Publication Nos. US 2009/0047263, US 2009/0068742, US 2009/0191159, US 2009/0227032, US 2009/0246875, and US 2009/0304646, the disclosures of which are incorporated herein by reference. Generally, to generate iPSCs, somatic cells are provided with reprogramming factors (e.g., Oct4, SOX2, KLF4, MYC, Nanog, Lin28, etc.) known in the art to reprogram the somatic cells to become pluripotent stem cells.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, e.g., ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

By “mitotic cell” it is meant a cell undergoing mitosis. Mitosis is the process by which a eukaryotic cell separates the chromosomes in its nucleus into two identical sets in two separate nuclei. It is generally followed immediately by cytokinesis, which divides the nuclei, cytoplasm, organelles and cell membrane into two cells containing roughly equal shares of these cellular components.

By “post-mitotic cell” is meant a cell that has exited from mitosis (is in Go), e.g., the cell is “quiescent,” e.g., it is no longer undergoing cell division. This quiescent state may be temporary, e.g., reversible, or it may be permanent.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, e.g., arresting its development; (c) relieving the disease, e.g., causing regression of the disease, or reducing the risk of disease or a symptom of a disease.

The therapeutic agent may be administered before, during, or after the onset of disease or injury. The treatment of ongoing disease, where the treatment stabilizes or reduces the undesirable clinical symptoms of the subject, is of particular interest. Such treatment is desirably performed prior to complete loss of function in affected tissues. In some cases, therapy is administered to a subject having at least on disease symptom. In some cases the treatment is administered after the subject is not experiencing one or more symptoms of the disease.

The terms “individual,” “subject,” “host,” and “patient,” are used interchangeably herein and refer to any mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., Cold Spring Harbor Laboratory Press 2001); Greenberg and Sambrook. Molecular Cloning: A Laboratory Manual, Fourth Edition, 2012, Cold Spring Harbor Laboratory Press; Short Protocols in Molecular Biology, 4th Ed. (Ausubel et al., eds., John Wiley & Sons 1999); Protein Methods (Bollag et al., John Wiley & Sons 1996); Nonviral Vectors for Gene Therapy (Wagner et al., eds., Academic Press 1999); Viral Vectors (Kaplift & Loewy eds., Academic Press 1995); Immunology Methods Manual (1. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### EXAMPLES

### Example 1: Bacillus Subtilis Upp/5-FU Assay System

Described herein is the Bacillus subtilis upp gene and knockout mutations resulting in resistance to 5-fluorouracil (5-FU) as a system to characterize mutation rates and mutation types induced by random mutagenesis, according to the invention.

The B. subtilis (substrain 168) upp gene (NCBI Reference Sequence: NC_000964.3, https://www.ncbi.nlm.nih.gov/nuccore/NC_000964.3; as retrieved on May 29, 2020) encodes an uracil phosphoribosyltransferase enzyme which is a pyrimidine salvage enzyme. This enzyme also converts 5-fluorouracil directly to 5-fluorouridine monophosphate, a very potent inhibitor of thymidylate synthetase (Neuhard, 1983). As a result, culturing B. subtilis on plates supplemented with 5-FU causes toxicity for all cells expressing a functioning upp gene and selection for cells lacking a functional copy (Fabret, 2002). This makes upp a useful target for detecting very low levels of mutagenesis, with a variety of potential mutations (e.g., additions, deletions, substitutions) that will result in selectable loss-of-function. The small size of this gene (630 bp, 210 aa) allows for PCR amplicon sequencing and rapid analysis of numerous potential mutations. It should be noted that in the upp gene is inessential when ample uracil is supplied.

ROS induced mutations at upp: Reactive oxygen species vary in their reactivity and their mechanism of mutagenesis. Singlet oxygen has been demonstrated to induce a broad spectrum of mutations including single-nucleotide variants and chromosomal deletions (Noma, 2012). The most frequent product of oxidative damage to DNA is 8-oxo-2′-deoxyguanosine, which preferentially pairs with adenine rather than cytosine, resulting in G:C→T:A base pair transversions. Any out-of-frame insertion/deletion mutation in the first 441 nt of the upp gene will result in a premature stop before the C-terminal active site that would result in upp loss-of-function. There are 21 potential G:C→T:A base pair transversions that would result in a premature stop before the C-terminal active site (provided in Table 1).

TABLE 1
Predicted G:C→T:A transversions within the
first 486 nt of upp resulting in premature stop.

| Resulting Stop Codon | WT Codon, AA (n) | | |
|---|---|---|---|
| TGA | GGA, G (5) | TGC, C (0) | |
| TAA | GAA, E (10) | TCA, S (1) | TAC, Y (2) |
| TAG | GAG, E (2) | TCG, S (1) | |

In a preliminary sequence analysis of 94 selected 5-FU-resistant mutants, 68 of the sequenced mutants (72%) exhibited G:C→T:A base pair transversions.

### Example 2: Generating Guided ROSProduction Strains of B. subtilis Str 168

Identified upp target sites for the RNA-guided DNA-binding protein dLbaCpf1: Five guide sequences were chosen to target dLbaCpf1 to the upp gene of B. subtilis. As off-target controls, five guide sequences were chosen to target the amyE gene of B. subtilis (NCBI Reference Sequence: NC_000964.3, https://www.ncbi.nlm.nih.gov/nuccore/NC_000964.3). For both sets, target sequences were chosen for their predicted score using DeepCpf1 (Kim, 2018). Also, targets were designed to eliminate effects of CRISPR-induced inhibition (CRISPRi) by targeting the nontemplate strand (Seong Keun Kim, Haseong Kim, Woo-Chan Ahn, Kwang-Hyun Park, Eui-Jeon Woo, Dae-Hee Lee, and Seung-Goo Lee, ACS Synthetic Biology 2017 6 (7), 1273-1282, DOI: 10.1021/acssynbio.6b00368).

Guide expression constructs: An array of guide expression vectors was constructed with a synthetic, broad-spectrum constitutive promoter driving a series of direct repeats and 23-bp targeting sequences terminated by a T7 terminator.

All guide expression cassettes were inserted into pBV70, a modified derivative of pMiniMAD2 (Patrick & Kearns, 2008) between BamHI and EcoRI restriction sites. These plasmids included selectable markers conferring resistance to the antibiotics ampicillin (for E. coli cloning) and erythromycin (for B. subtilis maintenance), origins from pBR322 (for E. coli cloning) and temperature-sensitive pE194 (for B. subtilis maintenance), and a mobilization fragment (mob) to allow bacterial conjugation.

ROSProducing Cas-protein fusion constructs: An array of dLbaCpf1 expression plasmids were constructed with different ROSProducing fusions, connected by a short flexible linker. These fusion products (and one plasmid without a fused protein) included nuclear localization sequences at either end and were driven by a synthetic, broad-spectrum constitutive promoter and terminated by a T7 terminator.

All dLbCpf1 expression plasmids included selectable markers conferring resistance to the antibiotic kanamycin, and origins from pBR322 (for E. coli cloning) and temperature-sensitive pE194 (for B. subtilis maintenance).

### Example 3: Guided ROS Mutagenesis of Upp Gene in the Presence of Catalytically Inactive RNA-Guided Endonucleases and Upp Guide RNAs

To test the rate and spectrum of mutations induced by guided ROSProduction, an experiment was performed where B. subtilis str. 168 cells were co-transformed with combinations of guide expression and fusion dLbaCpf1 expression plasmids (Table 2).

TABLE 2
Combinations of guide expression and fusion dLbaCpf1
expression plasmids used for this experiment.

| Strain | | SEQ ID | Guide | SEQ ID |
|---|---|---|---|---|
| Name | Fusion dLbaCpf1 plasmid | NO: | Plasmid | NO: |
| 0353 | pBV003 | 3 | pBV053 | 5 |
| | (dLbaCpf1-mOrange2) | | (3X upp) | |
| 3341 | pBV033 | 4 | pBV041 | 6 |
| | (dLbaCpf1-Pp2FbFP_L30M) | | (amyE) | |
| 3353 | pBV033 | 4 | pBV053 | 5 |
| | (dLbaCpf1-Pp2FbFP_L30M) | | (3X upp) | |

Light-induced mutagenesis treatment: A single colony for each plasmid combination was inoculated into LB medium supplemented with lincomycin (25 mg/L), kanamycin (5 mg/L) and erythromycin (1 mg/L) and cultured overnight at 30° C. Overnight cultures were diluted 25-fold into fresh selective media and arrayed into 24-well blocks for incubation at 30° C. with agitation and with or without illumination cycling (15 min on, 1 hr off) over 24 hours.

Determining 5-FU resistant counts: Cultures were diluted 10-fold into LB and 100 μL were plated in triplicate onto LB agar plates containing 6.5 mg/L 5-FU to quantify resistant CFU. After a 24-hour incubation at 37° C., resistant CFU were counted.

To determine total viable count, treated cultures were further diluted to 10⁵ in LB and plated onto LB agar plates to quantify total CFU. After an overnight incubation at 30° C., total CFU were counted. The results of this experiment are demonstrated by rate of resistant CFU in FIG. 1. In this experiment, targeting the upp gene resulted in a 27-fold increase in rate of resistant CFU relative to targeting amyE gene. Fusion to Pp2FbFP_L30M resulted in an 11-fold increase in rate of resistant CFU relative to mOrange2 fusion. Light cycling resulted in a 22-fold increase in rate of resistant CFU relative to dark treatment.

### Example 4: Characterizing the Position and Frequency of Mutations Induced by Guided ROSProduction

To understand the types of mutations introduced by guided ROSProduction, the upp regions of 5-FU-resistant colonies were amplified by PCR and sequenced.

Mutational analysis by Sanger sequencing: In one experiment, a guide expression construct containing an array of three guides targeting the B. subtilis upp gene (SEQ ID NO: 5) was transformed into B. subtilis str. 168 harboring a plasmid expressing dead LbaCpf1 fused to either mOrange2 or Pp2FbFP_L30M (SEQ ID NOs: 3, 4). A single colony for each was inoculated into LB medium supplemented with lincomycin (25 mg/L), kanamycin (5 mg/L) and erythromycin (1 mg/L) and cultured overnight at 30° C. Overnight cultures were diluted 25-fold into fresh selective media and arrayed into 24-well blocks for incubation at 30° C. with agitation and illumination cycling (15 min on, 1 hr off) over 24 hours. Cultures were diluted 10-fold into LB and 100 μL were plated in triplicate onto LB agar plates containing 6.5 mg/L 5-FU. After a 24-hour incubation at 37° C., colonies were picked into 150 μL of sterile water and microwaved for 4 minutes to lyse the cells. The upp region was amplified by PCR (SEQ ID NOs: 7, 8) and sequenced using nested primers (SEQ ID NOs: 9, 10). The upp gene was sequenced for 27 colonies with mOrange2 and 57 colonies with Pp2FbFP_L30M. The results of the sequencing analysis are provided below in Table 3.

Observed mutations to upp in B. subtilis resulting in functional knockout and resistance to 5-FU. Positions are provided with reference to the upp coding sequence.

TABLE 3
Results of sequencing analysis

| | | | | | | | | | | | 590- |
|---|---|---|---|---|---|---|---|---|---|---|---|
| | Position | 247 | 401 | 404 | 540 | 545 | 547 | 548 | 556 | 579 | 671 |
| mOrange2 | G:C → A:T | 0 | 0 | 1 | 0 | 0 | 0 | 0 | 0 | 0 | 0 |
| | A:T → G:C | 0 | 1 | 0 | 0 | 0 | 0 | 0 | 0 | 0 | 0 |
| | G:C → T:A | 0 | 0 | 1 | 12 | 6 | 0 | 0 | 1 | 0 | 0 |
| | C ↔ G | 0 | 0 | 0 | 2 | 0 | 1 | 0 | 0 | 0 | 0 |
| | T ↔ A | 0 | 0 | 0 | 0 | 0 | 0 | 0 | 0 | 1 | 0 |
| | DEL | 0 | 0 | 0 | 0 | 0 | 0 | 0 | 0 | 0 | 1 |
| Pp2FbFP_L30M | G:C → A:T | 1 | 0 | 0 | 0 | 0 | 0 | 0 | 0 | 0 | 0 |
| | A:T → G:C | 0 | 0 | 0 | 0 | 0 | 0 | 0 | 0 | 0 | 0 |
| | G:C → T:A | 0 | 0 | 0 | 21 | 30 | 0 | 0 | 1 | 0 | 0 |
| | C ↔ G | 0 | 0 | 0 | 3 | 0 | 0 | 1 | 0 | 0 | 0 |
| | T ↔ A | 0 | 0 | 0 | 0 | 0 | 0 | 0 | 0 | 0 | 0 |
| | DEL | 0 | 0 | 0 | 0 | 0 | 0 | 0 | 0 | 0 | 0 |

More than 90% of the 57 sequenced upp fragments from 5-FU-resistant colonies generated in Pp2FbFP_L30M fusion strains had G:C→T:A transversion mutations present. These caused premature stops (C540A and G556T) and an A→E substitution (C554A).

### Example 5: Testing Several Potential ROSProducing Proteins for Guided Upp Mutagenesis

An experiment was designed to test different potential ROSProducing proteins for their ability to deliver guided mutagenesis. Tested proteins included fluorescent proteins (SuperNova, tagRFP, mOrange2) and flavin-mononucleotide-binding (Fb) proteins (SOPP3, Pp2FbFP_L30M, and an experimental chimera of the two). The variety of chosen proteins was predicted to produce differing levels of superoxide and singlet oxygen reactive oxygen species, with different photon efficiencies.

TABLE 4
dLbaCpf1 fusion expression vectors generated to test
the mutagenic effects of different ROS producers.

| Fusion Protein | SEQ ID NO: |
|---|---|
| mOrange2 | 3 |
| tagRFP | 11 |
| SuperNova | 12 |
| SOPP3 | 13 |
| Pp2FbFP_L30M | 4 |
| Chimera | 14 |

This experiment was completed for mOrange2, tagRFP, SOPP3, and Pp2FbFP_L30M, and the results are in FIG. 2.

Total viable CFU and resistant CFU counts for light and dark treatments of each plasmid combination. Resistant CFU relative to total CFU were calculated, and the standard deviation of triplicate plates is provided.

This experiment was completed for the FbFP chimera, SuperNova, Pp2FbFP-L30M, and Pp2FbFP_L30M with guide targeting amyE as a control.

Total viable CFU and resistant CFU counts for light and dark treatments of each plasmid combination. Resistant CFU relative to total CFU were calculated, and the standard deviation of triplicate plates is provided in FIG. 3.

### Example 6: Testing the Effect of Multiple Guides on Increasing ROS-Induced Mutagenesis

Sequence analysis of 5-FU-resistant strains was using three different guides directed to upp suggested that most mutations occurred at or near one target more frequently than the other two target sites. We designed an experiment to determine if the three guides together worked synergistically to generate greater localized mutagenesis than individually combined.

Generating guide expression constructs to test individual guide sequences and their combinations: We constructed several guide expression vectors to express guides for this experiment (Table 5).

TABLE 5
Guide expression vectors generated to test the
synergistic effect of multiple proximal guides.

| Guide(s) | SEQ ID NO: |
|---|---|
| amyE1 | 15 |
| amyE2 | 6 |
| upp1 | 16 |
| upp2 | 17 |
| upp3 | 18 |
| 3X upp | 5 |

All guide constructs were tested in combination with the dLbaCpf1-Pp2FbFP_L30M expression construct. A single colony for each plasmid combination was inoculated into LB medium supplemented with lincomycin (25 mg/L), kanamycin (5 mg/L) and erythromycin (1 mg/L) and cultured overnight at 30° C. Overnight cultures were diluted 25-fold into fresh selective media and arrayed into 24-well blocks for incubation at 30° C. with agitation and with or without illumination cycling (15 min on, 1 hr off) over 24 hours. Cultures were diluted 10-fold into LB and 100 μL were plated in triplicate onto LB agar plates containing 6.5 mg/L 5-FU to quantify resistant CFU. After a 24-hour incubation at 37° C., resistant CFU were counted. To determine total viable count, treated cultures were further diluted to 10⁵ in LB and plated onto LB agar plates to quantify total CFU. After an overnight incubation at 30° C., total CFU were counted.

The resulting chart summarizing the rate of resistant CFU is provided in FIG. 4. In this experiment, targeting the upp gene with three guides resulted in a 1.8-fold increase in rate of resistant CFU relative to the sum of the rates of all three individually expressed guides. When resistance rate was calculated as increase relative to dark controls (rate in light/rate in dark−1), the three guides resulted in an 3.1-fold increase compared to the sum of the increases from all three individually expressed guides.

### Example 7: Deep Amplicon Sequencing of Guided ROS-Induced Mutagenesis

Amplicon sequencing allows for useful read coverage of very many pooled mutants. We will generate a series of pools of both 5-FU-resistant mutants and unselected culture over repeated 24-hour light cycling experiments with dilution into selective LB for each round. We will test the strains shown in Table 6.

TABLE 6
The repeated exposure series will be
performed using the listed strains.

| Strain | Fusion | Guides |
|---|---|---|
| WT | NA | NA |
| Un-fused | None | 5X upp (SEQ ID NO: 19) |
| mOrange2 | mOrange2 | 5X upp |
| Pp2FbFP_L30M | Pp2FbFP_L30M | 5X upp |
| Non-targeted | Pp2FbFP_L30M | 5X amyE (SEQ ID NO: 20) |

A single colony for each plasmid combination was inoculated into LB medium supplemented with lincomycin (25 mg/L), kanamycin (5 mg/L) and erythromycin (1 mg/L) and cultured overnight at 30° C. Overnight cultures were diluted 25-fold into fresh selective media and arrayed into 24-well blocks for incubation at 30° C. with agitation and with or without illumination cycling (15 min on, 1 hr off) over 24 hours.

Sequential light cycling treatments to increase rate of guided ROS-induced mutagenesis: After each 24-hour round of light cycling treatment, 50 μL from each well is used to inoculate 950 μL of LB supplemented with lincomycin (25 mg/L), kanamycin (5 mg/L) and erythromycin (1 mg/L) for another round of light cycling. Also after each round, 100 μL of the treated culture is diluted 10-fold and 10⁵× and 100 μL is plated on 5-FU-supplemented LB agar plates and standard LB agar plates to quantify 5-FU resistant CFU and total CFU, respectively. Colonies are counted after a 24-hour incubation at 37° C. for the 5-FU plates and at 30° C. for the standard plates. After quantification, the plates are flooded with LB and scraped. These cell suspensions are transferred to culture tubes and incubated overnight at 30° C. Following this, genomic DNA is extracted from these mixed cultures and sent to Köln for deep amplicon sequencing.

### Example 8: Testing in Bacillus subtilis with Different Genetic Target (pyrF or rpoB)

To verify that this technique works beyond the selected upp gene target, the process is adapted for pyrF gene (5-FOA resisresistance) or rpoB gene (Rifampin resistance).

### Example 9: Targeted Mutagenesis for Functional Selection

This example describes combining catalytically inactive programmable DNA cleavage enzymes with DNA base modifying chemical mutagens to enrich mutagenesis in targeted regions of the enzyme 5-enolpyruvylshikimate-3-phosphate synthase (EPSPS).

EPSPS is the enzyme that catalyzes the conversion of phosphoenolpyruvate and 3-phosphoshikimate to phosphate and EPSPS. This enzyme is inhibited by the competitive inhibitor glyphosate, which is used widely in agriculture as an herbicide. The structure of EPSPS has been determined and single point mutants within the active site that overcome glyphosate inhibition have been identified. Bacterial screens with E. coli have been developed that allow for selection of improved EPSPS variants in the presence of glyphosate (see Jin et al., Curr. Microbiol. 55:350 (2007)). Variant EPSPS enzymes have been generated by multiple methods, including untargeted methods such as error prone PCR or targeted approaches that are expensive and require highly-skilled researcher inputs to develop designs and molecular biology skills for saturation mutagenesis libraries.

DNA base modifying chemical mutagens in combination with catalytically inactive CRISPR associated protein/guide RNA complexes coupled together with activity selection, such as in a bacterial EPSPS functional selection assay, to enrich mutagenesis to a selected region of the enzyme EPSPS.

Cpf1 or Cas9 gRNAs targeting a specific region of the EPSPS enzyme, such as the residues lining the active site, are designed. This may require a synthetic gene containing proper PAM sites at the desired location(s).

E. coli expressing the EPSPS gene is transformed with dLbCpf1 or dSpCas9 and cognate gRNAs. The transformed cells are subsequently treated with a EMS, and mutagenized cells are placed under selection by glyphosate. Mutations accumulate at higher rates in the targeted region of EPSPS, and when placed under selection by glyphosate the recovery of resistance-conferring mutations derived from the targeted residues is increased.

### Example 10: In Planta Targeted Gene Modification

Random chemical mutagenesis approaches to enhancing genetic diversity in plants requires balancing multiple factors for finding mutations in a candidate gene that include, mutation rate, viability and sterility after treatment, population size, the window of sequence evaluation, and others.

As the mutation rate decreases, the number of individuals required to screen to find a desired mutation increases exponentially. The local mutation rate induced by DNA base modifying chemical mutagens can be increased by utilizing sequence targeting enzymes (e.g., catalytically inactive RNA-guided endonuclease enzymes such as dLbCpf1 and dSpCas9). Once local mutation rates are increased, the number of individuals that need to be screened to find a desired mutation is reduced.

To enable this approach, the catalytically inactivated RNA-guided endonucleases and guide RNAs need to be present in the nucleus of a plant cell that will be treated with chemical mutagens. The catalytically inactivated RNA-guided endonuclease, gRNA, and EMS are titrated following standard procedures in the art to establish an initial kill-curve analysis for the dose and exposure times leading to a defined mortality (typically, 50% mortality is used in the art).

Targeted modification can be accomplished in multiple ways, including by expressing a catalytically inactivated RNA-guided endonuclease (e.g., dLbCpf1, dSpCas9) within the plant cell, either by co-delivering DNA or mRNA encoding the catalytically inactivated RNA-guided endonuclease or via stable transformation of the plant cells with the catalytically inactive RNA-guided endonuclease enzymes and/or gRNA. Following expression of the catalytically inactivated RNA-guided endonuclease and gRNA, EMS is applied using standard methods to induce targeted modifications of the target site.

An alternative approach for delivering a catalytically inactivated RNA-guided endonuclease and gRNA complex is to deliver the complex transiently as a ribonucleoprotein, which can be performed on a range of tissue types including leaves, pollen, protoplast, embryos, callus, and others. Following or concurrently with delivery, EMS is applied using standard methods to induce targeted modifications of the target site.

A number of seeds or regenerated plants are grown and screened for mutations in the targeted window using standard methods known in the art.

### Example 12: Increasing Accessibility of DNA-Damaging Chemistries for Therapeutic Treatments

Direct chemical modification of DNA to interfere with normal DNA replication has been shown to be effective in cancer therapy. Cancer cells have relaxed DNA damage sensing/repair capabilities, which helps them achieve high replication rates and also makes them more susceptible to DNA damage.

The replication of damaged DNA increases the probability of cell death and has been the focus for anticancer compound development. The DNA alkylating-like platinum compound Cis-diamminedichloroplatinum(II) (cisplatin) forms DNA adducts with guanine and, to a lesser extent, adenine residues. When two platinum adducts form on adjacent bases on the same DNA strand they form instrastrand crosslinks. These intrastrand crosslinks block DNA replication and cause cell death if not repaired (see Cheung-Ong et al., Chem. Biol., 20:648-59 (2013)). These therapies are not without side effects and discovery efforts for cisplatin analogs are directed to reducing toxicity in nontargeted tissues (see Bruijnincx and Sadler, Curr. Opin. Chem. Biol., 12:197-206 (2008)).

The compositions and methods described herein may be used to increase the effectiveness of a non-targeted chemical DNA modifying therapeutic treatment. Not wishing to be bound by a particular theory, the DNA bases of essential genes can be made more available for chemical modification by the unwinding action of catalytically inactivated RNAguided endonuclease/guide complexes that unwind the DNA. The delivery of catalytically inactivated RNA-guided endonuclease/guide complexes to target cancer cells is an active area of development and routes for selective targeting of tumor cells could include, but not limited to oncolytic viruses or microinjection. These routes could be used for selective delivery of catalytically inactivated RNA-guided endonucleases (see Liu et al., J Control Release, 266:17-26 (2017)). The combined effect of selectively unwinding and making available the DNA targeted residues (by exposing the targeted base from within the more protected dsDNA helix) for chemical modification in cancerous cells may lower the total dosage of DNA damaging chemotherapeutic required to induce cell death in cancer cells. By lowering the total dosage of chemical therapeutic required, the adverse toxicity to non-target tissues would be expected to be reduced.

### LEGEND TO FIGURES

FIG. 1: A plot of the rate of 5-FU resistant CFU relative to total CFU for each tested plasmid combination in Example 3, in both light cycled illumination (white bars) and dark conditions (black bars). Error bars represent standard deviation of triplicate samples.

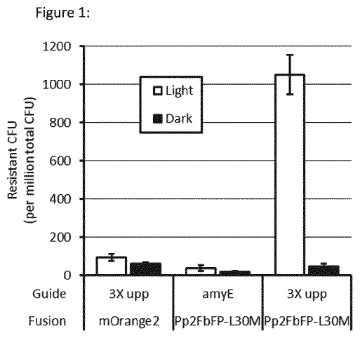

FIG. 2: The relative resistance rates of tested fusion cargos in Example 5: light cycled illumination (white bars) and dark conditions (black bars). Error bars represent standard deviation of triplicate samples.

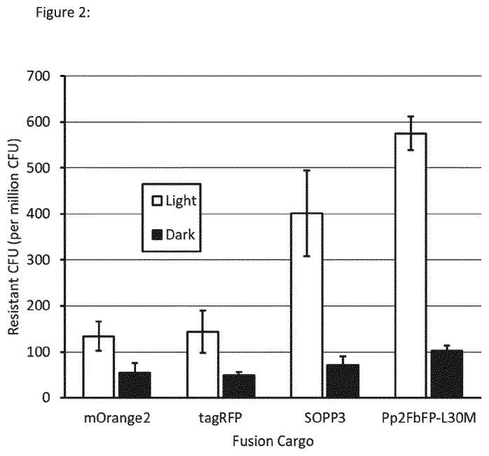

FIG. 3: The relative resistance rate of tested fusion cargos in Example 5, including an off-target control with Pp2FbFP_L30M targeted to amyE; light cycled illumination (white bars) and dark conditions (black bars). Error bars represent standard deviation of triplicate samples.

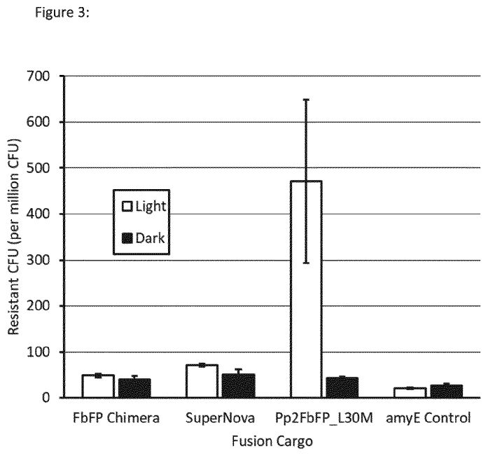

FIG. 4: The relative resistance rates of the tested guide combinations in Example 6; light cycled illumination (white bars) and dark conditions (black bars). Error bars represent standard deviation of triplicate samples.

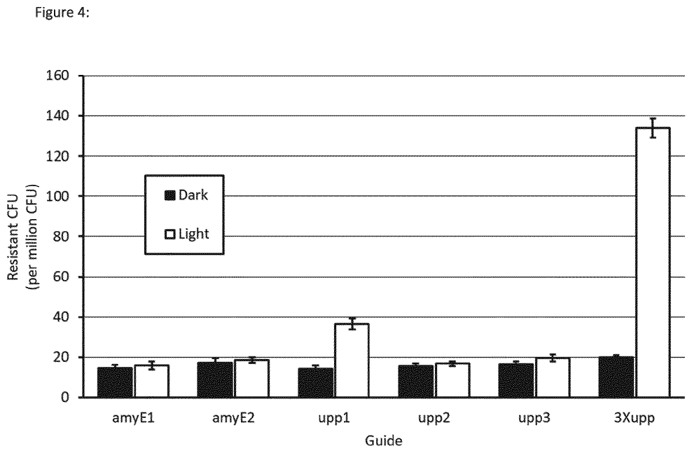

List of Sequences

| SEQ ID | | |
|---|---|---|
| NO: | Identifier | Remarks |
| 1 | dLbaCpf1_mOrange2_NA | dLbaCpf1 fused to mOrange2 nucleic acid |
| | | sequence |
| 2 | dLbaCpf1_mOrange2_AA | dLbaCpf1 fused to mOrange2 amino acid |
| | | sequence |
| 3 | pBV003dLbaCpf1_mOrange2_NA | pBV003 plasmid encoding dLbaCpf1 fused to |
| | | mOrange2 fluorescent protein nucleic acid |
| | | sequence |
| 4 | pBV033dLbaCpf1_Pp2FbFP_L30M_NA | pBV033 plasmid encoding dLbaCpf1 fused to |
| | | Pp2FbFP_L30M nucleic acid sequence |
| 5 | pBV053_3Xupp_guides_NA | pBV053 plasmid encoding guide array with 3 |
| | | upp guides nucleic acid sequence |
| 6 | pBV041_amyE2_guide_NA | pBV041 plasmid encoding amyE guide nucleic |
| | | acid sequence |
| 7 | pcruppfor_NA | Forward primer used to amplify upp |
| | | region from B. subtilis 168 |
| 8 | pcrupprev_NA | Reverse primer used to amplify upp |
| | | region from B. subtilis 168 |
| 9 | sequppfor_NA | Forward primer used to sequence upp |
| | | region from B. subtilis 168 |
| 10 | sequpprev_NA | Reverse primer used to sequence upp |
| | | region from B. subtilis 168 |
| 11 | pBV030dLbaCpf1_SuperNova_NA | pBV030 plasmid encoding dLbaCpf1 fused to |
| | | SuperNova nucleic acid sequence |
| 12 | pBV031dLbaCpf1_tagRFP_NA | pBV031 plasmid encoding dLbaCpf1 fused to |
| | | tagRFP nucleic acid sequence |
| 13 | pBV032dLbaCpf1_SOPP3_NA | pBV032 plasmid encoding dLbaCpf1 fused to |
| | | SOPP3 nucleic acid sequence |
| 14 | pBV034dLbaCpf1_Chimera_NA | pBV034 plasmid encoding dLbaCpf1 fused to |
| | | chimera nucleic acid sequence |
| 15 | pBV040_amyE1_guide_NA | pBV040 plasmid encoding amyE guide nucleic |
| | | acid sequence |
| 16 | pBV049_upp1_guide_NA | pBV049 plasmid encoding upp guide nucleic |
| | | acid sequence |
| 17 | pBV050_upp2_guide_NA | pBV050 plasmid encoding upp guide nucleic |
| | | acid sequence |
| 18 | pBV051_upp3_guide_NA | pBV051 plasmid encoding upp guide nucleic |
| | | acid sequence |
| 19 | pBV055_5Xupp_guides_NA | pBV055 plasmid encoding guide array with 5 |
| | | upp guides nucleic acid sequence |
| 20 | pBV054_5XamyE_guides_NA | pBV054 plasmid encoding guide array with 5 |
| | | amyE guides nucleic acid sequence |
| 21 | dLbaCpf1 fused to Pp2FbFPL_30M amino | dLbaCpf1 fused to Pp2FbFPL_30M amino acid |
| | acid sequence | sequence, open reading frame of plasmid under |
| | | SEQ ID NO: 4 |

1. A method for inducing one or more modifications in a target nucleic acid molecule, comprising the steps:
a) contacting the target nucleic acid molecule with a fusion protein comprising:
i) a nucleic acid recognition module (NARM);
ii) a protein that generates reactive oxygen species (ROSP);
iii) an optional a linker peptide between the NARM and the ROSP;
b) in the presence of a guide RNA complementary to one strand of the target nucleic acid molecule, and
c) an activation of the ROSP.

2. A method according to claim 1, wherein the
NARM is a catalytically inactive guided-nuclease, and in the presence of a guide RNA complementary to one strand of the target nucleic acid molecule, and an activation of the ROSP by illumination with light of an appropriate wavelength.

3. A method according to claim 2, wherein the
NARM is a catalytically inactive guided-nuclease, selected from Cas12a, preferably dLbaCpf1, and the ROSP is selected from mOrange2 or Pp2FbFP_L30M.

4. A method according to claim 1, wherein the
i) NARM is dLbaCpf1,
ii) ROSP is mOrange2 fluorescent protein or Pp2FbFP_L30M
and in the presence of a guide RNA complementary to one strand of the target nucleic acid molecule, and an activation of the ROSP by illumination with light of an appropriate wavelength to obtain a sufficient excitation of the ROSP.

5. A method according to any of the claims 1 to 4, wherein the modification is carried out in a prokaryotic or eukaryotic cell.

6. A method according to any of the claims 1 to 5, wherein the modification is carried out in a plant.

7. A method according to claim 5, wherein the wherein the modification is carried out in a prokaryotic cell.

8. A method according to claim 7, wherein the wherein the prokaryotic cell is of the genus Bacillus.

9. A method according to claim 7, wherein the wherein the prokaryotic cell is a strain of Bacillus subtilis.

10. A recombinant protein comprising
i) dLbaCpf1;
ii) mOrange2 fluorescent protein or Pp2FbFP_L30M.

11. A system comprising:
(i) a fusion protein according to any of the claims 1 to 4, and 9,
(ii) a guide RNA (gRNA) or nucleic acid encoding the gRNA,
wherein the components (i) and (ii) are cloned into a appropriate plasmid which allows expression in the host cell.


SEQUENCE LISTING (21 sequences)

SEQ ID NO: 1 - DNA, 4463 bp, Artificial Sequence
  dLbaCpf1 fused to mOrange2 nucleic acid sequence
tgggtagcaaaaagaggcgtatcaagcaggacagcaaactggaaaaattcaccaactgttactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgttgcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatgtttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgattgccacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaatgcccgaaaaacatcttcaaaatcaacaccgaagtgcgtgttctgctgaaacacgatgataacccgtacgttattggcatcgcccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatctgtgaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgtgcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgtgcctgaccagcgcgtataaagaactgttcaacaaatacggcattaactaccagcagggcgacattcgtgcgctgctgtgtgaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaagcatgcaggatctggagtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaagggatctaagaagcgtaggatcaagcaagattga

SEQ ID NO: 2 - protein, 1487 aa, Artificial Sequence
  dLbaCpf1 fused to mOrange2 amino acid sequence
MGSKKRRIKQDSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHAGSGVSKGEENNMAIIKEFMRFKVRMEGSVNGHEFEIEGEGEGRPYEGFQTAKLKVTKGGPLPFAWDILSPHFTYGSKAYVKHPADIPDYFKLSFPEGFKWERVMNYEDGGVVTVTQDSSLQDGEFIYKVKLRGTNFPSDGPVMQKKTMGWEASSERMYPEDGALKGKIKMRLKLKDGGHYTSEVKTTYKAKKPVQLPGAYIVDIKLDITSHNEDYTIVEQYERAEGRHSTGGMDELYKGSKKRRIKQD

SEQ ID NO: 3 - DNA, 7521 bp, Artificial Sequence
  pBV003 plasmid encoding dLbaCpf1 fused to mOrange2 fluorescent protein nucleic acid sequence
tctgtacaggcgcgccctaatttgacagtagaattagcatgtgatataataaataatttttactacccaagcttataaaagagcactgttgggcgtgagtggaggcgccggaaaaaagcatcgaaaaaaggaggaaaaaaacatatgggtagcaaaaagaggcgtatcaagcaggacagcaaactggaaaaattcaccaactgttactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgttgcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatgtttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgattgccacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaatgcccgaaaaacatcttcaaaatcaacaccgaagtgcgtgttctgctgaaacacgatgataacccgtacgttattggcatcgcccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatctgtgaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgtgcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgtgcctgaccagcgcgtataaagaactgttcaacaaatacggcattaactaccagcagggcgacattcgtgcgctgctgtgtgaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaagcatgcaggatctggagtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaagggatctaagaagcgtaggatcaagcaagattgagcggccgctgagcaataactagcataaccccttggggcctctaaacgggtcttgaggggttttttgctgaaacctcagggaattcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaaatgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtctgcagttgaaatcccctcaaaaacccgatataatgggtttaatagctgaataagaacggtgctctccaaatattcttatttagaaaagcaaatctaaaattatctgaaaggggaatgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtgcaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctcgagctctgagagatcccctcataatttcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggccattc

SEQ ID NO: 4 - DNA, 7276 bp, Artificial Sequence
  pBV033 plasmid encoding dLbaCpf1 fused to Pp2FbFP_L30M nucleic acid sequence
tctgtacaggcgcgccctaatttgacagtagaattagcatgtgatataataaataatttttactacccaagcttataaaagagcactgttgggcgtgagtggaggcgccggaaaaaagcatcgaaaaaaggaggaaaaaaacatatgggtagcaaaaagaggcgtatcaagcaggacagcaaactggaaaaattcaccaactgttactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgttgcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatgtttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgattgccacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctawgatgtatataaagatwaacgtttctccgaagatcagtacgaactgcacatcccgatcgcaattaacaaatgcccgaaaaacatcttcaaaatcaacaccgaagtgcgtgttctgctgaaacacgatgataacccgtacgttattggcatcgcccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatctgtgaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgtgcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgtgcctgaccagcgcgtataaagaactgttcaacaaatacggcattaactaccagcagggcgacattcgtgcgctgctgtgtgaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaagcatgcaggatctggaggtggagggtctatgatcaatgcaaaactgctgcagctgatggttgaacatagcaatgatggtattgttgttgcagaacaagaaggcaacgaaagcattatgatttatgtgaatccggcatttgaacgcctgaccggttattgtgcagatgatattctgtatcaggatgcacgttttctgcagggtgaagatcacgatcagcctggtattgcaattattcgtgaagcaattcgcgaaggtcgtccgtgttgtcaggttctgcgtaattatcgtaaagatggtagcctgttttggaacgaactgagcattacaccggttcataatgaagcagatcagctgacctattacattggtattcagcgtgatgttaccgcacaggtttttgccgaagaacgtgttcgtgaactggaagcagaagttgccgaactgcgtcgtcagcagggtcaagcaaaacatggatctaagaagcgtaggatcaagcaagattgagcggccgctgagcaataactagcataaccccttggggcctctaaacgggtcttgaggggttttttgctgaaacctcagggaattcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaaatgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtctgcagttgaaatcccctcaaaaacccgatataatgggtttaatagctgaataagaacggtgctctccaaatattcttatttagaaaagcaaatctaaaattatctgaaaggggaatgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtgcaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctcgagctctgagagatcccctcataatttcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggccattcgcca

SEQ ID NO: 5 - DNA, 6755 bp, Artificial Sequence
  pBV053 plasmid encoding guide array with 3 upp guides nucleic acid sequence
tcgtgatacgcctatttttataggttaatgtcatgataataatggtttcttagaggtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgcaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccctccttgacactgaatttagcatgtgatataattaacttaatattctacccaagcttataaaagagcactgttgggcgtgagtggaggcgccggaaaaaagcatcgaaaaaaaatttctactaagtgtagatatctagcgccgcaatgtaaatataatttctactaagtgtagatgcagcctgaaccggtgtattgataatttctactaagtgtagataaatgccgtcaaccattcccaataatttctactaagtgtagatggcagcgatgcatcgctgcgaatttctactaagtgtagattgaagagcgctcttctaatttctactaagtgtagatttttcaattgcatgaagaatctgcttacataaccccttggggcctctaaacgggtcttgaggggttttttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagcggccgcaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattcgtttttaggaaatttaaactgcaatatatccttggtaaaaactttgaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacacatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcaatggatcagtacaagaaagatactgtatttcataaacaggaactgcaagaagttaaggatgagttacagaaggcaaataagcagttacagagtggaatagagcacatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagatagtacagtgactttatatcgtgctgcgagggcgaatttccctgggtttgagaaagggtttaataggcttaaagagaaattctttaatgattccaaattcgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcacccttataatatcatgagcgaatatgtaataaaagaaactgaaaacaagaaaaattcaagatgacttaattggaaatttgttttagaaccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaagagtgtcacaagacactcttttttcgcaccaacgaaaactggtttaagccgactgcgctcga

SEQ ID NO: 6 - DNA, 6739 bp, Artificial Sequence
  pBV041 plasmid encoding amyE guide nucleic acid sequence
tcgtgatacgcctatttttataggttaatgtcatgataataatggtttcttagaggtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgcaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccctccttgacactgaatttagcatgtgatataattaacttaatattctacccaagcttataaaagagcactgttgggcgtgagtggaggcgccggaaaaaagcatcgaaaaaaaatttctactaagtgtagatatgccgccaaacatatagagcttaatttctactaagtgtagatgggtcttcgaagaccgaatttctactaagtgtagattgagacgcgtctctaatttctactaagtgtagatggcagcgatgcatcgctgcgaatttctactaagtgtagattgaagagcgctcttctaatttctactaagtgtagatttttcaattgcatgaagaatctgcttacataaccccttggggcctctaaacgggtcttgaggggttttttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagcggccgcaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattcgtttttaggaaatttaaactgcaatatatccttggtaaaaactttgaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacacatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcaatggatcagtacaagaaagatactgtatttcataaacaggaactgcaagaagttaaggatgagttacagaaggcaaataagcagttacagagtggaatagagcacatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagatagtacagtgactttatatcgtgctgcgagggcgaatttccctgggtttgagaaagggtttaataggcttaaagagaaattctttaatgattccaaattcgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcacccttataatatcatgagcgaatatgtaataaaagaaactgaaaacaagaaaaattcaagatgacttaattggaaatttgttttagaaccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaagagtgtcacaagacactcttttttcgcaccaacgaaaactggtttaagccgactgcgctcga

SEQ ID NO: 7 - DNA, 19 bp, Bacillus subtilis
  Forward primer used to amplify upp region from B. subtilis 168
tccgggcaaacgctactac

SEQ ID NO: 8 - DNA, 19 bp, Bacillus subtilis
  Reverse primer used to amplify upp region from B. subtilis 168
gccaagaagttagcccctg

SEQ ID NO: 9 - DNA, 18 bp, Bacillus subtilis
  Forward primer used to sequence upp region from B. subtilis 168
ggttttttgacgatgttc

SEQ ID NO: 10 - DNA, 25 bp, Bacillus subtilis
  Reverse primer used to sequence upp region from B. subtilis 168
attttcacctataattgtatacaga

SEQ ID NO: 11 - DNA, 7545 bp, Artificial Sequence
  pBV030 plasmid encoding dLbaCpf1 fused to SuperNova nucleic acid sequence
tctgtacaggcgcgccctaatttgacagtagaattagcatgtgatataataaataatttttactacccaagcttataaaagagcactgttgggcgtgagtggaggcgccggaaaaaagcatcgaaaaaaggaggaaaaaaacatatgggtagcaaaaagaggcgtatcaagcaggacagcaaactggaaaaattcaccaactgttactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgttgcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatgtttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgattgccacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaatgcccgaaaaacatcttcaaaatcaacaccgaagtgcgtgttctgctgaaacacgatgataacccgtacgttattggcatcgcccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatctgtgaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgtgcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgtgcctgaccagcgcgtataaagaactgttcaacaaatacggcattaactaccagcagggcgacattcgtgcgctgctgtgtgaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaagcatgcaggatctggaggtggagggtctatgggtagcgaagttggtccggcactgtttcagagcgatatgacctttaaaatctttatcgatggcgaagtgaacggccagaaatttaccattgttgcagatggtagcagcaaatttccgcatggtgatttcaatgttcatgccgtttgtgaaaccggtaaactgccgatgagctggaaaccgatttgtcatctgattcagtatggcgaaccgttttttgcacgttatccggatggtattagccattttgcacaagaatgttttccggaaggtctgagcattgatcgtaccgttcgttttgaaaatgatggcaccatgaccagccatcatacctatgaactggatgatacctgtgttgttagccgtattaccgttaattgtgatggttttcagccggatggtccgattatgcgtgatcagctggttgatattctgccgagcgaaacccatatgtttccacatggtccgaatgcagttcgtcagaccgcaaccattggttttaccaccgcagatggtggcaagatgatgggtcattttgatagcaagatgacgtttaatggtagccgtgcaattgaaattccgggtccgcattttgttaccattattaccaaacagacccgtgataccagcgataaacgtgatcatgtttgtcagcgtgaagttgcatatgcccatagcgttccgcgtattaccagcgcaattggtagtgatgaagatggatctaagaagcgtaggatcaagcaagattgagcggccgctgagcaataactagcataaccccttggggcctctaaacgggtcttgaggggttttttgctgaaacctcagggaattcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaaatgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtctgcagttgaaatcccctcaaaaacccgatataatgggtttaatagctgaataagaacggtgctctccaaatattcttatttagaaaagcaaatctaaaattatctgaaaggggaatgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtgcaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctcgagctctgagagatcccctcataatttcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggccattc

SEQ ID NO: 12 - DNA, 7617 bp, Artificial Sequence
  pBV031 plasmid encoding dLbaCpf1 fused to tagRFP nucleic acid sequence
tctgtacaggcgcgccctaatttgacagtagaattagcatgtgatataataaataatttttactacccaagcttataaaagagcactgttgggcgtgagtggaggcgccggaaaaaagcatcgaaaaaaggaggaaaaaaacatatgggtagcaaaaagaggcgtatcaagcaggacagcaaactggaaaaattcaccaactgttactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgttgcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatgtttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgattgccacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaatgcccgaaaaacatcttcaaaatcaacaccgaagtgcgtgttctgctgaaacacgatgataacccgtacgttattggcatcgcccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatctgtgaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgtgcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgtgcctgaccagcgcgtataaagaactgttcaacaaatacggcattaactaccagcagggcgacattcgtgcgctgctgtgtgaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaagcatgcaggatctggaggtggagggtctatggtgagcaaaggtgaggaactgatcaaagaaaacatgcatatgaaactgtacatggaaggcaccgtgaataaccatcactttaaatgtaccagcgaaggtgaaggtaaaccgtatgaaggcacccagaccatgcgtattaaagttgttgaaggtggtccgctgccgtttgcatttgatattctggcaaccagctttatgtatggtagccgtacctttattaaccatacccagggtattccggatttctttaaacagagctttccggaaggttttacctgggaacgtgttaccacctatgaagatggtggtgttctgaccgcaacccaggataccagtctgcaggatggttgtctgatttataatgtgaaaattcgcggtgtgaactttccgagcaatggtccggttatgcagaaaaaaaccctgggttgggaagcaaataccgaaatgctgtatccggcagatggtggcctggaaggtcgtagcgatatggcactgaaactggttggtggtggtcatctgatttgtaactttaaaaccacctatcgcagcaaaaaacctgccaaaaatctgaaaatgcctggcgtgtattatgtggatcatcgtctggaacgcattaaagaagcagataaagaaacctatgtggaacagcatgaagttgcagttgcacgttattgtgatctgccgagcaaactgggtcataaactgaatagcggtctgcgtagccgtgcacaggcaagcaatagcgcagttgatggcaccgcaggtccgggtagcaccggtagtcgcggatctaagaagcgtaggatcaagcaagattgagcggccgctgagcaataactagcataaccccttggggcctctaaacgggtcttgaggggttttttgctgaaacctcagggaattcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaaatgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtctgcagttgaaatcccctcaaaaacccgatataatgggtttaatagctgaataagaacggtgctctccaaatattcttatttagaaaagcaaatctaaaattatctgaaaggggaatgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtgcaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctcgagctctgagagatcccctcataatttcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggccattc

SEQ ID NO: 13 - DNA, 7143 bp, Artificial Sequence
  pBV032 plasmid encoding dLbaCpf1 fused to SOPP3 nucleic acid sequence
tctgtacaggcgcgccctaatttgacagtagaattagcatgtgatataataaataatttttactacccaagcttataaaagagcactgttgggcgtgagtggaggcgccggaaaaaagcatcgaaaaaaggaggaaaaaaacatatgggtagcaaaaagaggcgtatcaagcaggacagcaaactggaaaaattcaccaactgttactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgttgcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatgtttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgattgccacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaatgcccgaaaaacatcttcaaaatcaacaccgaagtgcgtgttctgctgaaacacgatgataacccgtacgttattggcatcgcccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatctgtgaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgtgcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgtgcctgaccagcgcgtataaagaactgttcaacaaatacggcattaactaccagcagggcgacattcgtgcgctgctgtgtgaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaagcatgcaggatctggaggtggagggtctgaaaaaagcttcgttattaccgatccgcgtctgccggataatccgatcatttttgcaagtgatggttttctggaactgaccgaatatagccgtgaagaaattctgggtcgtaatggtcgttttctgcagggtcctgaaaccgatcaggcaaccgttcagaaaattcgtgatgccattcgtgatcagcgtgaaattaccgttcagctgattaactataccaaaagcggcaaaaaattcctgaatctgctgaacctgcagccgattcgcgatcagaaaggtgaactgcaggcatttattggtgttgttctggatggtggatctaagaagcgtaggatcaagcaagattgagcggccgctgagcaataactagcataaccccttggggcctctaaacgggtcttgaggggttttttgctgaaacctcagggaattcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaaatgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtctgcagttgaaatcccctcaaaaacccgatataatgggtttaatagctgaataagaacggtgctctccaaatattcttatttagaaaagcaaatctaaaattatctgaaaggggaatgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtgcaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctcgagctctgagagatcccctcataatttcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggccattc

SEQ ID NO: 14 - DNA, 7272 bp, Artificial Sequence
  pBV034 plasmid encoding dLbaCpf1 fused to chimera nucleic acid sequence
tctgtacaggcgcgccctaatttgacagtagaattagcatgtgatataataaataatttttactacccaagcttataaaagagcactgttgggcgtgagtggaggcgccggaaaaaagcatcgaaaaaaggaggaaaaaaacatatgggtagcaaaaagaggcgtatcaagcaggacagcaaactggaaaaattcaccaactgttactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgttgcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatgtttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgattgccacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaatgcccgaaaaacatcttcaaaatcaacaccgaagtgcgtgttctgctgaaacacgatgataacccgtacgttattggcatcgcccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatctgtgaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgtgcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgtgcctgaccagcgcgtataaagaactgttcaacaaatacggcattaactaccagcagggcgacattcgtgcgctgctgtgtgaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaagcatgcaggatctggaggtggagggtctatgatcaatgcaaaactgctgcagctgatggttgaacattcaaatgatagcattgtggtgaccgaacaagaaggcaacgaaagcattatgatttatgtgaatccgggttttgaacgcctgaccggttattgtgcagatgatattctgtatcaggatggtcgttttctgcagggtgaagatcacgatcagcctggtattgcaattattcgtgaagcaattcgcgaaggtcgtccgtgttgtcaggttctgcgtaattatcgtaaagatggtagcctgtttctgaacgaactgaatattaccccgattcataatgaagcagatcagctgaccgcatatattggtattgttcgtgatgttaccgcacaggtttttgccgaagaacgtgttcgtgaactggaagcagaagttgcagaactgcgtcgtcagcagggtcaagcaaaacatggatctaagaagcgtaggatcaagcaagattgagcggccgctgagcaataactagcataaccccttggggcctctaaacgggtcttgaggggttttttgctgaaacctcagggaattcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaaatgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtctgcagttgaaatcccctcaaaaacccgatataatgggtttaatagctgaataagaacggtgctctccaaatattcttatttagaaaagcaaatctaaaattatctgaaaggggaatgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtgcaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctcgagctctgagagatcccctcataatttcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggccattc

SEQ ID NO: 15 - DNA, 6739 bp, Artificial Sequence
  pBV040 plasmid encoding amyE guide nucleic acid sequence
tcgtgatacgcctatttttataggttaatgtcatgataataatggtttcttagaggtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgcaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccctccttgacactgaatttagcatgtgatataattaacttaatattctacccaagcttataaaagagcactgttgggcgtgagtggaggcgccggaaaaaagcatcgaaaaaaaatttctactaagtgtagatgccgaatatcacaaatacatctgaatttctactaagtgtagatgggtcttcgaagaccgaatttctactaagtgtagattgagacgcgtctctaatttctactaagtgtagatggcagcgatgcatcgctgcgaatttctactaagtgtagattgaagagcgctcttctaatttctactaagtgtagatttttcaattgcatgaagaatctgcttacataaccccttggggcctctaaacgggtcttgaggggttttttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagcggccgcaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattcgtttttaggaaatttaaactgcaatatatccttggtaaaaactttgaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacacatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcaatggatcagtacaagaaagatactgtatttcataaacaggaactgcaagaagttaaggatgagttacagaaggcaaataagcagttacagagtggaatagagcacatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagatagtacagtgactttatatcgtgctgcgagggcgaatttccctgggtttgagaaagggtttaataggcttaaagagaaattctttaatgattccaaattcgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcacccttataatatcatgagcgaatatgtaataaaagaaactgaaaacaagaaaaattcaagatgacttaattggaaatttgttttagaaccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaagagtgtcacaagacactcttttttcgcaccaacgaaaactggtttaagccgactgcgctcga

SEQ ID NO: 16 - DNA, 6739 bp, Artificial Sequence
  pBV049 plasmid encoding upp guide nucleic acid sequence
tcgtgatacgcctatttttataggttaatgtcatgataataatggtttcttagaggtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgcaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccctccttgacactgaatttagcatgtgatataattaacttaatattctacccaagcttataaaagagcactgttgggcgtgagtggaggcgccggaaaaaagcatcgaaaaaaaatttctactaagtgtagatatctagcgccgcaatgtaaatataatttctactaagtgtagatgggtcttcgaagaccgaatttctactaagtgtagattgagacgcgtctctaatttctactaagtgtagatggcagcgatgcatcgctgcgaatttctactaagtgtagattgaagagcgctcttctaatttctactaagtgtagatttttcaattgcatgaagaatctgcttacataaccccttggggcctctaaacgggtcttgaggggttttttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagcggccgcaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattcgtttttaggaaatttaaactgcaatatatccttggtaaaaactttgaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacacatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcaatggatcagtacaagaaagatactgtatttcataaacaggaactgcaagaagttaaggatgagttacagaaggcaaataagcagttacagagtggaatagagcacatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagatagtacagtgactttatatcgtgctgcgagggcgaatttccctgggtttgagaaagggtttaataggcttaaagagaaattctttaatgattccaaattcgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcacccttataatatcatgagcgaatatgtaataaaagaaactgaaaacaagaaaaattcaagatgacttaattggaaatttgttttagaaccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaagagtgtcacaagacactcttttttcgcaccaacgaaaactggtttaagccgactgcgctcga

SEQ ID NO: 17 - DNA, 6739 bp, Artificial Sequence
  pBV050 plasmid encoding upp guide nucleic acid sequence
tcgtgatacgcctatttttataggttaatgtcatgataataatggtttcttagaggtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgcaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccctccttgacactgaatttagcatgtgatataattaacttaatattctacccaagcttataaaagagcactgttgggcgtgagtggaggcgccggaaaaaagcatcgaaaaaaaatttctactaagtgtagatgcagcctgaaccggtgtattgataatttctactaagtgtagatgggtcttcgaagaccgaatttctactaagtgtagattgagacgcgtctctaatttctactaagtgtagatggcagcgatgcatcgctgcgaatttctactaagtgtagattgaagagcgctcttctaatttctactaagtgtagatttttcaattgcatgaagaatctgcttacataaccccttggggcctctaaacgggtcttgaggggttttttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagcggccgcaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattcgtttttaggaaatttaaactgcaatatatccttggtaaaaactttgaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacacatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcaatggatcagtacaagaaagatactgtatttcataaacaggaactgcaagaagttaaggatgagttacagaaggcaaataagcagttacagagtggaatagagcacatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagatagtacagtgactttatatcgtgctgcgagggcgaatttccctgggtttgagaaagggtttaataggcttaaagagaaattctttaatgattccaaattcgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcacccttataatatcatgagcgaatatgtaataaaagaaactgaaaacaagaaaaattcaagatgacttaattggaaatttgttttagaaccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaagagtgtcacaagacactcttttttcgcaccaacgaaaactggtttaagccgactgcgctcga

SEQ ID NO: 18 - DNA, 6739 bp, Artificial Sequence
  pBV051 plasmid encoding upp guide nucleic acid sequence
tcgtgatacgcctatttttataggttaatgtcatgataataatggtttcttagaggtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgcaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccctccttgacactgaatttagcatgtgatataattaacttaatattctacccaagcttataaaagagcactgttgggcgtgagtggaggcgccggaaaaaagcatcgaaaaaaaatttctactaagtgtagataaatgccgtcaaccattcccaataatttctactaagtgtagatgggtcttcgaagaccgaatttctactaagtgtagattgagacgcgtctctaatttctactaagtgtagatggcagcgatgcatcgctgcgaatttctactaagtgtagattgaagagcgctcttctaatttctactaagtgtagatttttcaattgcatgaagaatctgcttacataaccccttggggcctctaaacgggtcttgaggggttttttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagcggccgcaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattcgtttttaggaaatttaaactgcaatatatccttggtaaaaactttgaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacacatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcaatggatcagtacaagaaagatactgtatttcataaacaggaactgcaagaagttaaggatgagttacagaaggcaaataagcagttacagagtggaatagagcacatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagatagtacagtgactttatatcgtgctgcgagggcgaatttccctgggtttgagaaagggtttaataggcttaaagagaaattctttaatgattccaaattcgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcacccttataatatcatgagcgaatatgtaataaaagaaactgaaaacaagaaaaattcaagatgacttaattggaaatttgttttagaaccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaagagtgtcacaagacactcttttttcgcaccaacgaaaactggtttaagccgactgcgctcga

SEQ ID NO: 19 - DNA, 6765 bp, Artificial Sequence
  pBV055 plasmid encoding guide array with 5 upp guides nucleic acid sequence
tcgtgatacgcctatttttataggttaatgtcatgataataatggtttcttagaggtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgcaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccctccttgacactgaatttagcatgtgatataattaacttaatattctacccaagcttataaaagagcactgttgggcgtgagtggaggcgccggaaaaaagcatcgaaaaaaaatttctactaagtgtagatatctagcgccgcaatgtaaatataatttctactaagtgtagatgcagcctgaaccggtgtattgataatttctactaagtgtagataaatgccgtcaaccattcccaataatttctactaagtgtagatattccgtatatatgtcagcttgtaatttctactaagtgtagataaatgccatgagtgtagccacttaatttctactaagtgtagatttttcaattgcatgaagaatctgcttacataaccccttggggcctctaaacgggtcttgaggggttttttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagcggccgcaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattcgtttttaggaaatttaaactgcaatatatccttggtaaaaactttgaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacacatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcaatggatcagtacaagaaagatactgtatttcataaacaggaactgcaagaagttaaggatgagttacagaaggcaaataagcagttacagagtggaatagagcacatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagatagtacagtgactttatatcgtgctgcgagggcgaatttccctgggtttgagaaagggtttaataggcttaaagagaaattctttaatgattccaaattcgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcacccttataatatcatgagcgaatatgtaataaaagaaactgaaaacaagaaaaattcaagatgacttaattggaaatttgttttagaaccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaagagtgtcacaagacactcttttttcgcaccaacgaaaactggtttaagccgactgcgctcga

SEQ ID NO: 20 - DNA, 6765 bp, Artificial Sequence
  pBV054 plasmid encoding guide array with 5 amyE guides nucleic acid sequence
tcgtgatacgcctatttttataggttaatgtcatgataataatggtttcttagaggtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgcaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccctccttgacactgaatttagcatgtgatataattaacttaatattctacccaagcttataaaagagcactgttgggcgtgagtggaggcgccggaaaaaagcatcgaaaaaaaatttctactaagtgtagatagataggactgtacttgtgtattaatttctactaagtgtagatcccgggaacctcacaccatttccaatttctactaagtgtagatcctggctccaatgattcggatttaatttctactaagtgtagatgcggcatcaaatcgaaaaccgtcaatttctactaagtgtagatgaatactcttaacctcattggaaaatttctactaagtgtagatttttcaattgcatgaagaatctgcttacataaccccttggggcctctaaacgggtcttgaggggttttttgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagcggccgcaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattcgtttttaggaaatttaaactgcaatatatccttggtaaaaactttgaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacacatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcaatggatcagtacaagaaagatactgtatttcataaacaggaactgcaagaagttaaggatgagttacagaaggcaaataagcagttacagagtggaatagagcacatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagatagtacagtgactttatatcgtgctgcgagggcgaatttccctgggtttgagaaagggtttaataggcttaaagagaaattctttaatgattccaaattcgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcacccttataatatcatgagcgaatatgtaataaaagaaactgaaaacaagaaaaattcaagatgacttaattggaaatttgttttagaaccagaatcagcaaaagccgagtggttagagtatttaaaagagttacacattcaatttgtagtgtctccattacatgatagggatactgatacagaagataggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaagagtgtcacaagacactcttttttcgcaccaacgaaaactggtttaagccgactgcgctcga

SEQ ID NO: 21 - protein, 1404 aa, Artificial Sequence
  dLbaCpf1 fused to Pp2FbFPL_30M amino acid sequence
MGSKKRRIKQDSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHAGSGGGGSMINAKLLQLMVEHSNDGIVVAEQEGNESIMIYVNPAFERLTGYCADDILYQDARFLQGEDHDQPGIAIIREAIREGRPCCQVLRNYRKDGSLFWNELSITPVHNEADQLTYYIGIQRDVTAQVFAEERVRELEAEVAELRRQQGQAKHGSKKRRIKQD